SEQUENCE LISTING (14 sequences)

SEQ ID NO: 1 - DNA, 20 bp, Artificial Sequence
  Synthetic
attagataccctdgtagtcc

SEQ ID NO: 2 - DNA, 16 bp, Artificial Sequence
  Synthetic
taccttgttacgactt

SEQ ID NO: 3 - DNA, 19 bp, Artificial Sequence
  Synthetic
tccgtaggtgaacctgcgg

SEQ ID NO: 4 - DNA, 20 bp, Artificial Sequence
  Synthetic
tcctccgcttattgatatgc

SEQ ID NO: 5 - DNA, 26 bp, Artificial Sequence
  Synthetic
atcgaaacgcctgaaggtccaaacat

SEQ ID NO: 6 - DNA, 23 bp, Artificial Sequence
  Synthetic
acacccttgttaccgtgacgacc

SEQ ID NO: 7 - DNA, 21 bp, Artificial Sequence
  Synthetic
gatcgtcaagcagccttagat

SEQ ID NO: 8 - DNA, 20 bp, Artificial Sequence
  Synthetic
ttaccgaccataacgccgac

SEQ ID NO: 9 - DNA, 19 bp, Artificial Sequence
  Synthetic
agagttgatcctggctcag

SEQ ID NO: 10 - DNA, 16 bp, Artificial Sequence
  Synthetic
gggttgcgctcgttrc

SEQ ID NO: 11 - DNA, 24 bp, Artificial Sequence
  Synthetic
ttcccccgtctccacttcttcatg

SEQ ID NO: 12 - DNA, 24 bp, Artificial Sequence
  Synthetic
gacgagatcgttcatgttgaactc

SEQ ID NO: 13 - DNA, 20 bp, Artificial Sequence
  Synthetic
catcgagaagttcgagaagg

SEQ ID NO: 14 - DNA, 20 bp, Artificial Sequence
  Synthetic
tacttgaaggaacccttacc

(19) United States
(12) Patent Application Publication
(10) Pub. No.: US 2023/0413857 A1
(43) Pub. Date: Dec. 28, 2023

(54) COMPOSITIONS OF MICROORGANISMS AND METHODS OF USING SAME

(71) Applicant: The State of Israel, Ministry of Agriculture & Rural Development, Agricultural Research Organization, Rishon Lizion (IL)

(72) Inventors: David EZRA, Ein Vered (IL); Ynon DEUTSCH, Tel Aviv (IL); Lior GUR, Hatzor-Haglilit (IL)

(21) Appl. No.: 18/038,399

(22) PCT Filed: Nov. 24, 2021
(86) PCT No.: PCT/IL2021/051397

§ 371 Date: May 23, 2023

Related U.S. Application Data
(60) Provisional application No. 63/117,633, filed on Nov. 24, 2020.

(51) Int. Cl. A23K 10/18; A61K 35/741; A61K 35/748; A61K 36/06; A23K 50/80

(52) U.S. Cl. CPC A23K 10/18; A61K 35/741; A61K 35/748; A61K 36/06; A23K 50/80

(57) ABSTRACT

The present invention, in some embodiments thereof, is directed to an artificial composition including microorganisms, and a method of using same, such as for preventing or treating a pathogenic infection in an aquatic organism.

### CROSS-REFERENCE TO RELATED APPLICATIONS

This application claims the benefit of priority of U.S. Provisional Patent Application No. 63/117,633, titled “COMPOSITIONS OF MICROORGANISMS AND METHODS OF USING SAME”, filed Nov. 24, 2020, the contents of which are incorporated herein by reference in their entirety.

### FIELD OF INVENTION

The present invention, in some embodiments thereof, is in the field of microbiology, and is directed to methods of biological control.

### BACKGROUND

Endophytes are a group of endosymbiotic microorganisms, often bacteria or fungi, that colonizes inter/intra cellular locations of all plants in natural ecosystems. In these mutualistic relations the plant provides a living niche and food for the endophyte while the endophyte provides protection against pathogens, pests and induces tolerance to abiotic stresses such as drought, salinity and more. Some endophytes produce and secrete biologically active compounds that prevent bacteria, fungi and plant pests from growing in and/or on the host plant. These compounds are called “secondary metabolites” they are produced by the endophytes in an aim to protect their living niche. In recent years, endophytes and their secondary metabolites are increasingly recognized for their importance in medical and industrial uses as well as in producing new biological control agents for agricultural uses. Endophytes are also found in aquatic plants including algae. Several studies describe the isolation of fungal and bacterial endophytes and identification of various novel compounds with antibiotic activity from seaweeds.

For the last twenty years aquaculture is considered the fastest growing industry of agriculture for food purposes. From the year 1980 to 2000 the annual growth was 10 percent. Although since 2000 it declined to 5.8 percent, it still continued to grow faster than other major food production sectors. The need to find additional protein sources for food supply of the growing world population, has led to this remarkable increasing growth.

Pathogens are a major and harmful factor in the aquaculture industry of vertebrates and invertebrates around the world. They cause huge economical losses which exceed 6 billion US$ per year. In addition to the well-known viral diseases and parasites, diseases caused by bacteria, fungi, and oomycete are common (30% of all losses). Pesticides and antibiotics, including additional/other therapeutic agents, are widely used to cope with these pathogens. Previously, malachite green, a toxic and carcinogenic suspected chemical compound, was extensively used as the major chemical control against fungal diseases and parasites. It was banned for use, worldwide, since the year 2000, due to the discovery that the compound as well as its residue breakdown can be found in the fish flesh long after its marketing. Other chemicals are used in an attempt to control aquaculture diseases (e.g., formaldehyde, copper sulfate, chlorine and hydrogen peroxide), though there is a growing necessity to reduce the use of those toxic compounds that eventually may harm the end consumers, in addition to the environmental pollution they cause. Currently, the ability to control disease outbreaks in aquaculture is very limited to none.

### SUMMARY

According to a first aspect, there is provided an artificial composition comprising cultured microorganisms comprising at least one isolate listed in Table 2, and an agriculturally acceptable carrier.

According to another aspect, there is provided a method for preventing or treating a pathogenic infection in an aquatic organism, the method comprising contacting the aquatic organism with an effective amount of the artificial composition of the invention.

In some embodiments, the artificial composition further comprises one or more metabolites produced by the at least one isolate, secreted therefrom, or both.

In some embodiments, the one or more metabolites produced by the at least one isolate, secreted therefrom, or both, comprises: 8-Nonenoic acid, 4-cumylphenol, or both.

In some embodiments, the metabolite is 8-Nonenoic acid. In some embodiments, the metabolite is 4-cumylphenol. In some embodiments, the metabolites are 8-Nonenoic acid and 4-cumylphenol.

In some embodiments, the at least one isolate is selected from the group consisting of: AJr9, ABp5, PU9, PU10, HU4N5, AU4, AJr10, PSH6, PSH7, ABp4, PLp5, PP2, PP6, KM3, KM4, PAn4, PAc1, HGGCy9, HGM1, HG3N2, TAPaN3, TAEnN2, and TAAnN2.

In some embodiments, the at least one isolate is AJr9.

In some embodiments, the artificial composition further comprises one or more bacteria belonging to a genus selected from the group consisting of: Bacillus, Lysinibacillus, Pseudomonas, Staphylococcus, and any combination thereof.

In some embodiments, the artificial composition further comprises at least one fungus belonging to the genus Beauveria.

In some embodiments, the artificial composition further comprises an alga.

In some embodiments, the artificial composition is a dried or a lyophilized composition.

In some embodiments, the artificial composition is characterized by being capable of inhibiting a pathogen's growth, killing a pathogen, inhibiting the secretion, activity, or both, of metabolites derived from a pathogen, or any combination thereof.

In some embodiments, the pathogen is a microorganism.

In some embodiments, the pathogen is selected from the group consisting of: a bacterium, a fungus, and an oomycete.

In some embodiments, the pathogen is selected from the group consisting of: Photobacterium damselae subsp. Damselae, Streptococcus iniae, Aeromonas salmonicida, Saprolegnia parasitica, and any combination thereof.

In some embodiments, the pathogen is Photobacterium damselae subsp. Damselae and the at least one isolate is selected from the group consisting of: AJr3, AJr7, AJr9, AJr10, AJr15, PSH1, PSH3, PSH5, PSH6, ABp1, ABp2, ABp3, ABp4, PAc1, PAc2, TAAcN4, PLp2, PLp5, PLp6, PAn4, TAAnN1, TAAnN2, TAAnN3, PP1, PP2, PP3, PP6, PP7, PP8, TAPaN1, TAPaN2, TAPaN3, KM FWTA1, KM FWTA3, HG9, PU9, PU10, AU1, HU4N5, HG2N7, HG3N2, HG3M1, TAEnN2, TAEnN4, and any combination thereof.

In some embodiments, the pathogen is Streptococcus iniae and the at least one isolate is selected from the group consisting of: AJr3, AJr9, AJr10, AJr15, PSH1, PSH3, PSH5, PSH6, ABp1, ABp2, PAc1, PAc2, TAAcN4, PLp2, PLp5, PLp6, PAn4, TAAnN1, TAAnN2, TAAnN3, PP1, PP2, PP3, PP6, PP7, PP8, TAPaN1, TAPaN2, TAPaN3, KM FWTA1, KM FWTA3, FWTa5, PU9, PU10, AU1, AU4, HU4N5, HG2N7, HG3N2, HG3M1, and any combination thereof.

In some embodiments, the pathogen is Aeromonas salmonicida and the at least one isolate is selected from the group consisting of: AJr3, AJr7, AJr9, AJr10, PSH1, PSH3, PSH5, PSH6, PSH7, ABp1, ABp2, ABp3, ABp4, PAc1, PAc2, TAAcN4, PLp2, PLp5, PLp6, PAn4, TAAnN1, TAAnN2, TAAnN3, PP2, PP3, PP6, PP7, TAPaN1, TAPaN2, TAPaN3, KM FWTA1, KM FWTA3, FWTa1, FWTa5, HG9, PU10, AU1, AU4, HU4N5, HG2N7, HG3N2, HG3M1, TAEnN2, TAEnN4, and any combination thereof.

In some embodiments, the pathogen is Saprolegnia parasitica and the at least one isolate is selected from the group consisting of: AJr3, AJr7, AJr9, AJr10, AJr15, PSH1, PSH3, PSH5, PSH6, PSH7, ABp1, ABp2, ABp3, ABp4, ABp5, PAc1, PAc2, TAAcN4, PLp2, PLp5, PLp6, PP2, PP6, PP7, TAPaN3, TAPaN3, KM FWTA1, KM FWTA3, FWTA4, PU9, PU10, AU1, AU4, HG3M1, TAEnN2, and any combination thereof.

In some embodiments, the one or more metabolites is 8-Nonenoic acid, 4-cumylphenol, or a combination thereof.

In some embodiments, the artificial composition is a feed composition.

In some embodiments, the feed is suitable for an aquatic organism.

In some embodiments, the aquatic organism is an aquacultured organism.

In some embodiments, the aquatic organism is cultured in freshwater, brackish water, saline water, or any combination thereof.

In some embodiments, the organism is selected from the group consisting of: a fish, a crustacean, a mollusk, a macro-alga, a phytoplankton, phytoplankton and zooplankton.

In some embodiments, the contacting comprises contacting a body of water comprising the aquatic organism.

In some embodiments, the contacting comprises providing the artificial composition of the invention as feed to the aquatic organism.

In some embodiments, the feed comprises the artificial composition of the invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A1-D includes images showing the isolation and bioactivity assessment of endophytes from algae. Isolating the endophytes from treated algae pieces (A1-A2), then performing in vitro bio activity assays (B1-B2 and C1-C2), and a pathogen's viability test (D).

Endophytes tested for activity against oomycete pathogen: Saprolegnia parasitica (C2 and D right) and three bacterial pathogens: Photobacterium damselae, Streptococcus iniae and Aeromonas salmonicida (B2). Control plates; the pathogens in the absence of the endophytes (B1, C1 and D left).

FIG. 2 includes a vertical bar graph showing the bioactivity of the endophytes isolated from different algae. The endophytes isolated from the twenty different algae are classified by the level of their bioactivity (left bar). The highly active endophytes (HAE) are featured by their activity against each of the four pathogens (right bar).

FIG. 3 includes a Venn diagram of the endophytes active against four-aquaculture pathogens. Fifty highly active endophytes isolated from the different algae, sorted by their bioactivity against the four-aquaculture pathogens: three bacterial pathogens (A. salmonicida, S. iniae, and P. damselae) and one Oomicota (S. parasitica). There was identified one endophyte active against all four pathogens (indicated by a triangle). Four endophytes active only against S. parasitica were identified (indicated by a circle), and 11 endophytes active against bacterial pathogens were identified (indicated by a square).

FIG. 4 includes a vertical bar graph showing the bioactivity of the different isolated endophytes according to the algal source. Black bars represent the non-active endophytes while active and very active isolates are displayed as gray and white bars accordingly. X axis includes the algal host from which endophytes were isolated.

FIG. 5 includes a vertical bar graph showing Endophytes' activity against four aquaculture pathogens as the number of active endophytes in each alga specie collected. The number of active endophytes from certain alga can be greater than the sum of isolates from the same alga because the same isolate can be active against more than one pathogen. X axis includes the algal host from which endophytes were isolated.

FIG. 6 includes a vertical bar graph showing endophytes' bioactivity displayed in association with sampling location. Each bar represents the sum of all isolated endophytes from all the algae types collected in the mentioned site. The different colors in the bar represents the number of isolates with the marked activity against the four pathogens; black, gray and white bars represent the number of non-active, active and highly active endophytes, respectively, isolated at the sampling location. X axis includes the collection sites where algal hosts were collected.

FIG. 7 includes a graph showing principal component analysis (PCA) of highly active endophytes (HAE) isolation in regard to habitat, location, and algae genus. HAE isolation from the different algae genus is most influenced by the “habitat” (type of water: marine, fresh water or thermo-mineral). An influence of the location is noticeable. Component 1 is the “habitat”, component 2 is the HAE isolate rate. Different shapes represent algae genus. The numbers represent the location of algae sampling. Broken-line circles are the clustering by habitat (left marine, right thermo-mineral). Full-line circles are the clustering by location.

FIGS. 8A-8B include fluorescent micrographs showing green fluorescent protein (GFP) transmitted endophytes within algal tissue (8A). Negative control algal tissue (8B). In 8A: Left, GFP expression detection; middle, cell nuclei stain (propidium iodide); right, overlay of left and middle micrographs. In 8B: Left, GFP expression detection; right, cell nuclei stain (propidium iodide).

FIGS. 9A-9B include graphs showing GFP intensity of introduced endophytes to algae over time (9A), and CFU of endophytes from algae over time (9B).

FIG. 10 includes an image showing an in-vitro bioactivity assay of PU10GFP re-isolated from inoculated Ulva sp. against S. iniae.

FIG. 11 includes a vertical bar graph showing CFU of introduced endophytes from dried algae over time.

FIGS. 12A-12C include images showing in-vitro bioactivity assays of ABp5 against Saprolegnia parasitica. (12A) A two compartments petri dish assay; (12B) a one compartment petri dish assay; and 12C a viability test showing loss of viability of S. parasitica plugs taken from either 12A or 12B (12C left) compared to control (12C right).

FIGS. 13A-13B include an image and a graph showing ABp5's compounds assay against S. parasitica (13A). (13B) S. parasitica growth with different concentrations of 8-Nonenoic acid in agar and water.

FIGS. 14A-14C include images showing in-vivo bioactivity assays of the compound 8-Nonenoic acid against Saprolegnia parasitica in Tilapia eggs. (14A) No eggs survival in the presence of S. parasitica plugs; (14B) negative control eggs (no S. parasitica plugs); and (14C) eggs introduced with S. parasitica plugs and 10 μg/ml 8-Nonenoic acid.

FIGS. 15A-15B include images showing in-vitro bioactivity assays of 8-Nonenoic acid against oomycete pathogens. (15A) Pythium aphanidermatum plugs; and (15B) Achlya bisexualis.

FIGS. 16A-16D include images showing in-vitro bioactivity assays of 4-Cumylphenol against oomycete pathogens. (16A) S. parasitica; (16B) Pythium aphanidermatum; (16C) Phytophthora infestans; and (16D) Achlya bisexualis.

FIGS. 17A-17C include images showing in-vitro bioactivity assays of 4-Cumylphenol against aquatic bacterial pathogens. (17A) Photobacterium damselae; (17B) S. iniae; and (17C) Aeromonas salmonicida.

### DETAILED DESCRIPTION

In some embodiments, the present invention is directed to a composition of microorganisms and acceptable carriers, such as for use in inhibiting growth and/or activity of a pathogen.

In some embodiments, the composition is an artificial composition.

As used herein, the term “artificial” refers to the composition disclosed herein being produced by or synthesized by man. In some embodiments, artificial comprises “man-made”. In some embodiments, artificial is not or excludes a product of nature per se. In some embodiments, the artificial composition of the invention comprises microorganisms which undergone at least one processing step, e.g., cultured, therefore is not or excludes a composition of microorganisms as isolated from nature per se. In some embodiments, the term “artificial” refers to a composition of microorganisms, inclusive of at least one isolate listed in Table 2 which was cultured, grown, processed, manipulated, or any combination thereof, in-vitro.

The terms “artificial” and “synthetic” are used herein interchangeably.

In some embodiments, the composition comprises cultured microorganisms.

In some embodiments, cultured comprises culturing microorganisms as disclosed herein, on a substrate. In some embodiments, the substrate comprises a liquid substrate, a solid substrate, a semi solid substrate, a solidified substrate, a gel substrate, or any combination thereof.

In some embodiments, cultured or culturing comprises fermenting or fermentation.

In some embodiments, the composition comprises microorganisms, inclusive of at least one isolate listed in Table 2, which were cultured in conventional bioreactors, shake flasks, test tubes, microtiter dishes, and petri plates, or any equivalent thereof, that would be apparent to one of ordinary skill in the art of microbiology as suitable for culturing a microorganism, e.g., bacterium, fungus, etc. In some embodiments, culturing is carried out at a temperature, pH and oxygen content appropriate for the at least one isolate listed in Table 2. In some embodiments, culturing conditions are within the expertise of one of ordinary skill in the art.

According to some embodiments, there is provided an artificial composition comprising microorganisms, comprising at least one isolate listed in Table 2, hereinbelow. In some embodiments, the composition comprises one isolate listed in Table 2. In some embodiments, the composition comprises a plurality of isolates listed in Table 2.

As used herein, the term “plurality” refers to any integer equal to or greater than 2. In some embodiments, a plurality comprises at least 2, at least 3, at least 5, at least 7, at least 9, or at least 10, or any value and range therebetween. Each possibility represents a separate embodiment of the invention. In some embodiments, a plurality comprises 2-7, 3-9, 4-8, or 2-10. Each possibility represents a separate embodiment of the invention.

In some embodiments, the composition comprises an acceptable carrier. In some embodiments, the carrier is an agriculturally acceptable carrier. In some embodiments, the carrier is an aquaculturally acceptable carrier. In some embodiments, an agriculturally acceptable carrier comprises an aquaculturally acceptable carrier, an environmentally acceptable carrier, or both. In some embodiments, the carrier is an environmentally acceptable carrier. Such carriers can be any material that an animal, a plant or the environment to be treated can tolerate. In some embodiments, the carrier comprises any material, which can be added to the isolate of the invention or a composition comprising same without causing or having an adverse effect on the environment, or any species or an organism other than the pathogenic microorganism. Furthermore, the carrier must be such that the isolate or composition comprising same remains effective at controlling a pathogenic microorganism.

In some embodiments, the carrier is a liquid carrier. In some embodiments, the carrier is a solid carrier. In some embodiments, the carrier is an organism. In some embodiments, the carrier is in a powder form. In some embodiments, the carrier is in the form of pellets, e.g., such as feed pellets. In some embodiments, the carrier is or comprises an alga. In some embodiments, an alga comprises dried alga. In some embodiments, an alga comprises a powder of an alga. In some embodiments, an alga comprises a freeze-dried alga. In some embodiments, an alga comprises a lyophilized alga. In some embodiments, an alga comprises an alga lysate, an alga extract, any fraction thereof, or any combination thereof.

In some embodiments, the at least one isolate disclosed herein makes up at least 0.02%, at least 0.05%, at least 0.1%, at least 0.3%, at least 0.5%, at least 0.7%, at least 1%, at least 2%, at least 3.5%, at least 5%, at least 7%, at least 12%, at least 15%, at least 20%, at least 30%, at least 40%, at least 50%, at least 60%, at least 70%, at least 80%, at least 85%, at least 90%, at least 93%, at least 95%, at least 97%, at least 99%, or 100% of the bacteria, microorganisms, or both, of the composition, or any value and range therebetween. Each possibility represents a separate embodiment of the invention. In some embodiments, the at least one isolate disclosed herein makes up 0.015-0.25%, 0.05-0.8%, 0.1-1.5%, 0.4-2%, 1-3%, 2-8%, 3-12%, 10-23%, 20-70%, 30-90%, 45-95%, 30-50%, 25-90%, 40-85%, 80-100%, 85-99%, 90-100%, 93-97%, 95-99%, or 97-100% of the bacteria, microorganisms, or both, of the composition. Each possibility represents a separate embodiment of the invention.

In some embodiments, the at least one isolate disclosed herein has a relative abundance of at least 0.02%, at least 0.05%, at least 0.1%, at least 0.3%, at least 0.5%, at least 0.7%, at least 1%, at least 2%, at least 3.5%, at least 5%, at least 7%, at least 12%, at least 15%, at least 20%, at least 30%, at least 40%, at least 50%, at least 60%, at least 70%, at least 80%, at least 85%, at least 90%, at least 93%, at least 95%, at least 97%, at least 99%, or 100% of the bacteria, microorganisms, or both, of the composition, or any value and range therebetween. Each possibility represents a separate embodiment of the invention. In some embodiments, the at least one isolate disclosed herein has a relative abundance of 0.015-0.25%, 0.05-0.8%, 0.1-1.5%, 0.4-2%, 1-3%, 2-8%, 3-12%, 10-23%, 20-70%, 30-90%, 45-95%, 30-50%, 25-90%, 40-85%, 80-100%, 85-99%, 90-100%, 93-97%, 95-99%, or 97-100% of the bacteria, microorganisms, or both, of the composition. Each possibility represents a separate embodiment of the invention.

In some embodiments, the composition further comprises one or more metabolites produced by the at least one isolate disclosed herein. In some embodiments, the composition further comprising one or more metabolites secreted by the at least one isolate disclosed herein. In some embodiments, the composition further comprises one or more metabolites produced and secreted by the at least one isolate disclosed herein.

As used herein, the term “metabolite” refers to any intermediate or end product of metabolism. In some embodiments, a metabolite comprises an organic metabolite. In some embodiments, a metabolite comprises an inorganic metabolite. In some embodiments, a metabolite comprises an aromatic metabolite or a metabolite comprising an aromatic moiety. In some embodiments, a metabolite comprises a volatile organic compound (VOC). In some embodiments, a metabolite comprises an anti-microbial compound. In some embodiments, a metabolite comprises any compound produced, secreted, or both, from at least one isolate as disclosed herein that is capable of inhibiting growth of a microbe, killing a microbe, reducing pathogenic activity of a microbe, or any combination thereof.

In some embodiments, the composition further comprises one or more bacteria belonging to a genus selected from: Bacillus, Lysinibacillus, Pseudomonas, Staphylococcus, or any combination thereof.

In some embodiments, the composition further comprises at least one fungus belonging to the genus Beauveria.

In some embodiments, the at least one isolate is selected from: AJr9, ABp5, PU9, PU10, HU4N5, AU4, AJr10, PSH6, PSH7, ABp4, PLp5, PP2, PP6, KM3, KM4, PAn4, PAc1, HGGCy9, HGM1, HG3N2, TAPaN3, TAEnN2, or TAAnN2 (as disclosed in Table 2).

In some embodiments, the at least one isolate comprises or is AJr9.

In some embodiments, the composition further comprises an alga.

In some embodiments, an alga comprises alga-derived material.

In some embodiments, alga-derived material comprises: an extract, a homogenate, biomass, a fraction thereof, a portion thereof, a compound isolated therefrom, or any combination thereof.

In some embodiments, the algae is selected from: Jania rubens, Spyridia harvey, Bryopsis plumosa, Alsidium corallinum, Laurencia papillosa, Acanthophora najadiformis, Padina pavonica, Pithophora spp., Leptolyngbya sp., Ulva sp., Gracilaria sp., Enteromorpha ralfsii, or any combination thereof.

In some embodiments, the composition is a dried or a lyophilized composition.

In some embodiments, a composition comprising at least one isolate selected from: AJr3, AJr7, AJr9, AJr10, and AJr15, further comprises the alga Jania rubens.

In some embodiments, a composition comprising at least one isolate selected from: PSH1, PH3, PSH5, PSH6, and PSH7, further comprises the alga Spyridia harvey.

In some embodiments, a composition comprising at least one isolate selected from: ABp1, ABp2, ABp3, ABp4, and ABp5, further comprises the alga Bryopsis plumosa.

In some embodiments, a composition comprising at least one isolate selected from: PAc1, PAc2, and TAAcN4 further comprises the alga Alsidium corallinum.

In some embodiments, a composition comprising at least one isolate selected from: PLp2, PLp5, and PLp6 further comprises the alga Laurencia papillosa.

In some embodiments, a composition comprising at least one isolate selected from: Pan4, TAAnN1, TAAnN2, and TAAnN3 further comprises the alga Acanthophora najadiformis.

In some embodiments, a composition comprising at least one isolate selected from: PP1, PP2, PP3, PP6, PP7, PP8, TAPaN1, TAPaN2, and TAPaN3 further comprises the alga Padina pavonica.

In some embodiments, a composition comprising at least one isolate selected from: KM1, KM3, KM4, FWTa1, and FWTa5 further comprises an alga selected from the Pithophora spp.

In some embodiments, a composition comprising the isolate HG9 further comprises an alga selected from Leptolyngbya sp.

In some embodiments, a composition comprising at least one isolate selected from: PU9, PU10, AU1, AU4, and HU4N5 further comprises an alga selected from the Ulva sp.

In some embodiments, a composition comprising at least one isolate selected from: HG2N7, HG3N2, and HG3M1 further comprises an alga selected from Gracilaria sp.

In some embodiments, a composition comprising at least one isolate selected from: TAEnN2, and HG3M1 further comprises the alga Enteromorpha ralfsii.

In some embodiments, the composition is characterized by being capable of inhibiting a pathogen's growth, killing a pathogen, inhibiting the secretion, activity, or both, of metabolites derived from a pathogen, or any combination thereof.

In some embodiments, a metabolite(s) derived from a pathogen reduce the wellbeing of an organism or a host. In some embodiments, a metabolite(s) derived from a pathogen diverts homeostasis of an organism or a host. In some embodiments, a metabolite(s) derived from a pathogen reduces fitness of an organism or a host. In some embodiments, a metabolite(s) derived from a pathogen is involved in, propagates, induces, initiates, any equivalent thereof or any combination thereof, pathogenesis of an organism or a host.

In some embodiments, a pathogen is a microbe or a microorganism.

The terms “microbe” and “microorganism” are interchangeable and refer to any microscopic-scale organism. In some embodiments, a microorganism comprises a single cell microorganism. In some embodiments, a microorganism comprises a multiple cell microorganism.

In some embodiments, a multiple cell microorganism comprises a colony of cells, or similarly structured community of cells, as would be apparent to one of ordinary skill in the art of microbiology.

In some embodiments, a pathogen is selected from: a bacterium, a fungus, a virus, a cnidarian, sea lice, flatworm, or an oomycete.

In some embodiments, the pathogen is an endo-pathogen or an endoparasite.

In some embodiments, the pathogen is an ecto-pathogen or an ectoparasite.

In some embodiments, the pathogen is an ecto-endo parasite.

In some embodiments, a pathogen is selected from: Photobacterium damselae subsp.

Damselae, Streptococcus iniae, Aeromonas salmonicida, Saprolegnia parasitica, or any combination thereof.

In some embodiments, a pathogen is selected from: Photobacterium damselae subsp. Damselae, Streptococcus iniae, Aeromonas salmonicida, Saprolegnia parasitica, Aeromonas hydrophilia, Vibrio harvii, Vibrio alginolyticus, or any combination thereof.

In some embodiments, a pathogen is selected from: Aeromonas hydrophilia, Vibrio harvii, Vibrio alginolyticus, or any combination thereof.

In some embodiments, the pathogen is Photobacterium damselae subsp. Damselae and the at least one isolate is selected from: AJr3, AJr7, AJr9, AJr10, AJr15, PSH1, PSH3, PSH5, PSH6, ABp1, ABp2, ABp3, ABp4, PAc1, PAc2, TAAcN4, PLp2, PLp5, PLp6, PAn4, TAAnN1, TAAnN2, TAAnN3, PP1, PP2, PP3, PP6, PP7, PP8, TAPaN1, TAPaN2, TAPaN3, KM FWTA1, KM FWTA3, HG9, PU9, PU10, AU1, HU4N5, HG2N7, HG3N2, HG3M1, TAEnN2, TAEnN4, or any combination thereof.

In some embodiments, the pathogen is Streptococcus iniae and the at least one isolate is selected from: AJr3, AJr9, AJr10, AJr15, PSH1, PSH3, PSH5, PSH6, ABp1, ABp2, PAc1, PAc2, TAAcN4, PLp2, PLp5, PLp6, PAn4, TAAnN1, TAAnN2, TAAnN3, PP1, PP2, PP3, PP6, PP7, PP8, TAPaN1, TAPaN2, TAPaN3, KM FWTA1, KM FWTA3, FWTa5, PU9, PU10, AU1, AU4, HU4N5, HG2N7, HG3N2, HG3M1, or any combination thereof.

In some embodiments, the pathogen is Aeromonas salmonicida and the at least one isolate is selected from: AJr3, AJr7, AJr9, AJr10, PSH1, PSH3, PSH5, PSH6, PSH7, ABp1, ABp2, ABp3, ABp4, PAc1, PAc2, TAAcN4, PLp2, PLp5, PLp6, PAn4, TAAnN1, TAAnN2, TAAnN3, PP2, PP3, PP6, PP7, TAPaN1, TAPaN2, TAPaN3, KM FWTA1, KM FWTA3, FWTa1, FWTa5, HG9, PU10, AU1, AU4, HU4N5, HG2N7, HG3N2, HG3M1, TAEnN2, TAEnN4, or any combination thereof.

In some embodiments, the pathogen is Saprolegnia parasitica and the at least one isolate is selected from: AJr3, AJr7, AJr9, AJr10, AJr15, PSH1, PSH3, PSH5, PSH6, PSH7, ABp1, ABp2, ABp3, ABp4, ABp5, PAc1, PAc2, TAAcN4, PLp2, PLp5, PLp6, PP2, PP6, PP7, TAPaN3, TAPaN3, KM FWTA1, KM FWTA3, FWTA4, PU9, PU10, AU1, AU4, HG3M1, TAEnN2, or any combination thereof.

In some embodiments, a pathogen is selected from: Henneguya salminicola, Lepeophtheirus salmonis, a Caligus species, e.g., C. clemensi and C. rogercresseyi, Gyrodactylus salaris, Yersinia ruckeri, and infectious salmon anemia virus (ISAv).

In some embodiments, a pathogen is selected from: white spot syndrome virus (WSSV), yellow head virus (YHV), Taura syndrome virus (TSV), infectious hypodermal and hematopoietic necrosis virus (IHHNV), gill-associated virus (GAV), infectious myonecrosis virus (IMNV), and hepatopancreatic parvovirus (HPV).

In some embodiments, the composition disclosed herein is a feed composition. In some embodiments, the composition disclosed herein is formulated with or mixed with a feed. In some embodiments, the herein disclosed composition is mixed with a pre-formed feed. In some embodiments, the herein disclosed composition is mixed with a formed feed. In some embodiments, the herein disclosed composition and a pre-formed feed are mixed and subsequently shaped or molded into feed suitable aquacultured organism. In some embodiments, the feed is of a shape selected from: pellets, powder, flakes, chips, or lentils. In some embodiments, a feed comprises a dried feed, e.g., pellets, flakes, etc. In some embodiments, a feed comprises a liquid feed. In some embodiments, the feed is a live feed or previously living feed, e.g., Artemia nauplii, mature Artemia, squid, a rotifer (e.g., belonging to the phylum “Rotifera”), or any other live feed known to one of ordinary skill in the art.

In some embodiments, the feed is suitable for an aquacultured organism. In some embodiments, the feed is a probiotic feed. In some embodiments, the feed is suitable for supporting growth and wellbeing or health of an aquacultured organism. In some embodiments, the feed comprises nutrients essential for the growth of an aquacultured organism and probiotic agent(s), e.g., the at least one isolate of the invention, essential for inhibiting a pathogen of the aquacultured organism.

Feed suitable for aquacultured organisms are common and would be apparent to one of ordinary skill in the art.

In some embodiments, the composition is an active filtration composition. In some embodiments, the composition is formulated as an active material capable of actively filtering a body of water. In some embodiments, the composition removes, kills, inhibits the activity, reduces the amount, or any combination thereof, of a pathogen inhabiting a body of water, or any compound secreted therefrom.

In some embodiments, the composition is ingested by an aquacultured organism. In some embodiments, the composition is absorbed in the gastrointestinal tract or an aquacultured organism, or any equivalent organ capable of absorbing feed in an aquacultured organism. In some embodiments, the composition is filtered from a body of water comprising the aquacultured organism into the body of the aquacultured organism. In some embodiments, the composition is filtered from a body of water comprising the aquacultured organism through the gills of the aquacultured organism into the body of the aquacultured organism, or any equivalent organ capable of filtering water and/or gas in an aquacultured organism. In some embodiments, the composition is filtered from a body of water comprising the aquacultured organism through the gills of the aquacultured organism into the circulation of the aquacultured organism.

According to some embodiments, there is provided an antimicrobial composition comprising 8-Nonenoic acid. In some embodiments, there is provided an antimicrobial composition comprising 4-cumylphenol.

According to some embodiments, there is provided an antibacterial composition comprising 8-Nonenoic acid. In some embodiments, there is provided an antibacterial composition comprising 4-cumylphenol.

In some embodiments, the antimicrobial composition is devoid of: nano-Ag/TiO2, Zinc Pyrithione, or a combination thereof. In some embodiments, an antimicrobial composition being an anti-fungal composition is devoid of: nano-Ag/TiO2, Zinc Pyrithione, or a combination thereof. In some embodiments, the antimicrobial composition comprising 4-cumylphenol is devoid of: nano-Ag/TiO2, Zinc Pyrithione, or a combination thereof. In some embodiments, an antimicrobial composition comprising 4-cumylphenol and being an anti-fungal composition is devoid of: nano-Ag/TiO2, Zinc Pyrithione, or a combination thereof.

In some embodiments, antibacterial comprises bacteriostatic.

In some embodiments, the antimicrobial or the antibacterial composition inhibits or reduces: the growth rate, DNA replication rate, CFU, protein production rate, gene translation rate or expression level, any activity essential for a living cell, or any combination thereof, of a bacterium, fungus, or both (e.g., the “targeted microbe”), as disclosed herein.

In some embodiments, the antimicrobial or the antibacterial composition is formulated for marine or aquatic application or appliance.

In some embodiments, the antimicrobial or the antibacterial composition is formulated for terrestrial application or appliance.

In some embodiments, the antimicrobial or the antibacterial composition disclosed herein, is used in a method as disclosed herein.

In some embodiments, the method comprises applying the antimicrobial or the antibacterial composition disclosed herein in an efficient amount.

In some embodiments, reduces is by at least 5%, at least 15%, at least 25%, at least 50%, at least 75%, at least 95%, or by 100%, or any value and range therebetween. Each possibility represents a separate embodiment of the invention. In some embodiments, inhibits is 100% inhibition.

In some embodiments, reduces is by 5-40%, 15-80%, 10-95%, 50-99%, 7-80%, or 10-100%, or any value and range therebetween. Each possibility represents a separate embodiment of the invention.

In some embodiments, microbe targeted by the herein disclosed composition is a marine microbe. In some embodiments, microbe targeted by the herein disclosed composition is a terrestrial microbe. In some embodiments, the microbe targeted by the herein disclosed composition is a plurality of types of targeted microbe comprising a marine microbe, a terrestrial microbe, or a combination thereof.

In some embodiments, the antibacterial and/or the antimicrobial composition comprises 8-Nonenoic acid in an amount of at least 0.1 μg/ml, at least 0.5 μg/ml, at least 1 μg/ml, at least 2 μg/ml, at least 5 μg/ml, at least 10 μg/ml, at least 15 μg/ml, at least 20 μg/ml, at least 25 μg/ml, at least 50 μg/ml, or at least 100 μg/ml, or any value and range therebetween. Each possibility represents a separate embodiment of the invention.

In some embodiments, the antibacterial and/or the antimicrobial composition comprises 8-Nonenoic acid in an amount of 0.1 to 20 μg/ml, 0.5 to 50 μg/ml, 1 to 20 μg/ml, 15 to 90 μg/ml, 1 to 100 μg/ml, 20 to 80 μg/ml, or 1 to 40 μg/ml.

In some embodiments, the antibacterial and/or the antimicrobial composition comprises 4-cumylphenol in an amount of at least 0.1 μg/ml, at least 0.5 μg/ml, at least 1 μg/ml, at least 2 μg/ml, at least 5 μg/ml, at least 10 μg/ml, at least 15 μg/ml, at least 20 μg/ml, at least 25 μg/ml, at least 50 μg/ml, or at least 100 μg/ml, or any value and range therebetween. Each possibility represents a separate embodiment of the invention.

In some embodiments, the antibacterial and/or the antimicrobial composition comprises 4-cumylphenol in an amount of 0.1 to 20 μg/ml, 0.5 to 50 μg/ml, 1 to 20 μg/ml, 15 to 90 μg/ml, 1 to 100 μg/ml, 20 to 80 μg/ml, or 1 to 40 μg/ml.

According to some embodiments, there is provided a method for preventing or treating a pathogenic infection in an aquatic organism, the method comprising contacting the aquatic organism with an effective amount of the herein disclosed composition.

As used herein, the terms “treatment” or “treating” of a disease, disorder or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, alleviated symptoms of the disease, disorder or condition.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, “treating” comprises ameliorating and/or preventing.

In some embodiments, the aquatic organism is cultured in freshwater, brackish water, or marine water. In some embodiments, the aquatic organism is cultured in saline water. In some embodiments, the aquatic organism is cultured in brine water.

As used herein, the term “freshwater” refers to water having a salinity lower than 0.5 parts per thousand (ppt).

As used herein, the term “brackish water” refers to water having a salinity of at least 0.5 ppt and lower than 30 ppt.

As used herein, the terms “marine water” and “brine water” are interchangeable and refer to water having a salinity of 30-50 ppt.

Methods, kits, and devices for determining water salinity level are common and would be apparent to one of ordinary skill in the art. Non-limiting example includes, but is not limited to, the use of a refractometer.

In some embodiments, the organism comprises an aquatic organism. In some embodiments, the aquatic organism comprises an aquacultured organism. In some embodiments, the organism is selected from: a fish, a crustacean, a mollusk, a phytoplankton, or a zooplankton. In some embodiments, the organism comprises an alga.

As used herein, the term “phytoplankton” encompasses any autotrophic member of the plankton community of a body of water, e.g., an ocean.

As used herein, the term “zooplankton” encompasses any encompasses any heterotrophic member of the plankton community of a body of water, e.g., an ocean. In some embodiments, a zooplankton comprises a microscopic zooplankton. In some embodiments, a zooplankton comprises a macroscopic zooplankton.

In some embodiments, contacting comprises contacting a body of water comprising the aquatic organism.

In some embodiments, contacting comprises providing the herein disclosed composition as feed to the aquatic organism. In some embodiments, the provided composition is ingested by the aquatic organism, filtered by the aquatic organism, adheres to the outer surface of the body of the aquatic organism, or any combination thereof.

In some embodiments, the antimicrobial activity of the herein disclosed composition is exerted: inside the body of the aquatic organism, outside the body of the aquatic organism, or both.

In some embodiments, the antimicrobial activity of the herein disclosed composition targets an endo-pathogen (e.g., endoparasite), an ecto-pathogen (e.g., ectoparasite), or both.

In some embodiments, the feed comprises the herein disclosed composition.

Any concentration ranges, percentage range, or ratio range recited herein are to be understood to include concentrations, percentages, or ratios of any integer within that range and fractions thereof, such as one tenth and one hundredth of an integer, unless otherwise indicated.

Any number range recited herein relating to any physical feature, such as polymer subunits, size, or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials and Methods

### Macro-Algae Sampling

Macro-algae were collected from different water sources in Israel: (a) sea water: the Mediterranean Sea; (b) freshwater: the Sea of Galilee, and different springs and rivers, and (c) sulfuric water: Hamat Gader spring (Table 1). Algae were brought to the laboratory, for further analysis in sealed plastic boxes containing the ambient water, from the collection location. During transport, the boxes were kept in chilled environment (±25° C.). After arrival to the laboratory, algae were kept in the transport boxes with addition of air bobbling until used for endophytes isolation.

Isolation of Endophytes from Algae

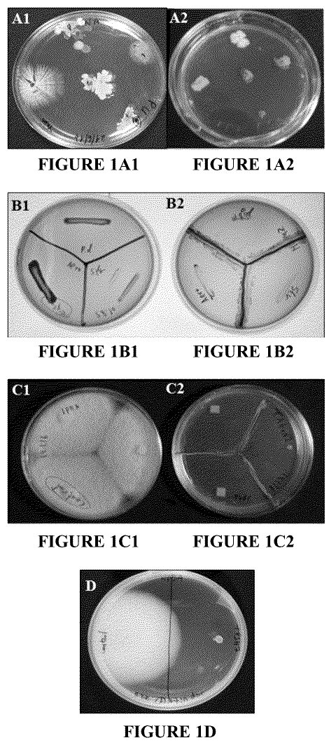

Endophytes isolation was performed under sterile conditions in a laminar hood. The sampled algae were washed with fresh water and algae surface were sterilized by washing algae pieces twice for three seconds in 70% (v/v) ethanol followed by two 1-minute washes in distilled water. To ensure algal surface sterilization; water from the alga's last wash were tested for bacterial or fungal presence by inoculation on appropriate agar media. No growth of bacteria or fungi confirms sterility of the algae's surface. The treated algae were cut to small pieces (˜0.5 cm by 0.5 cm) and placed on a growth media; Nutrient Agar (NA) for bacteria, Potato Dextrose Agar (PDA) for fungi (Acumedia, Michigan USA), in 90 mm petri plates. The petri plates were incubated at 25° C. for eight days, allowing the endophytes to grow out of the algae pieces (FIG. 1A). Bacteria and fungi growing during this time were collected and re-inoculated on appropriate growth media. A single colony/spore culture was established for the different endophytes, to ensure purity of the cultures.

### Bio Activity Assays

Endophytic bacteria and fungi were tested for biological activity against major aquaculture pathogens, three bacterial pathogens (Photobacterium damselae, Streptococcus iniae, and Aeromonas salmonicida) and one oomycote pathogen (Saprolegnia parasitica). In-vitro assays were performed on a synthetic media supporting both the endophytes and the pathogens; Tryptic Soy Agar (TSA, Acumedia, Michigan USA) for bacteria, PDA for fungi and NPDA (1/2 NA+1/2 PDA) concocted in the inventors' laboratory, for both fungi and bacteria in the same plate as follows: the tested endophytes were inoculated in a “Y shape” on the appropriate growth media in a 90 mm petri dish. The plates were incubated at 25° C. for 5 to 7 days. Then, the pathogens were introduced to the plates by inoculation at the edges of the plate. The plates were then incubated for another week (25° C.) and examined for pathogen's growth during that period (FIGS. 1B-1C). S. parasitica growth was examined by measuring the growth radius of the oomycete colony compared to the growth of a colony in the absence of the endophyte (control). Challenged bacterial pathogens' growth was estimated qualitatively using four levels of inhibition compared to control, as elaborated in the legend of Table 2.

### Viability Assays

Pathogens fully inhibited by the endophytes were tested for viability in order to verify whether they were killed or only inhibited by the endophyte. Viability assay was performed by re-culturing the inhibited pathogen from the bioassay plates onto a new growth media plate in the absence of the endophyte and incubating it under optimal growth conditions for a week. Usually, inhibited pathogens grew a few days later while dead pathogens did not grow new colonies (FIG. 1D).

### Identification of the Endophytes

Identification of endophytes was performed to the genus level by amplification, alignment of ribosomal DNA conserved sequences (ITS for fungi and 16S for bacteria) and comparing them to the gene bank. Identification to the species level was done by sequencing of at least three different conserved sequences as described.

### DNA Extraction

Single colony cultures of bacterial endophytes were incubated overnight at 25° C., 150 rpm, in Luria-Bertani broth (LB) media (Acumedia, Michigan USA). The next day, cultures were centrifuged, and 100 mg of pellet (wet weight) was used for the extraction using a fungal/bacterial DNA extraction kit according to the manufacture protocol (ZYMO RESEARCH, California USA). Pure cultures of fungal endophytes were incubated on PDA plates for four days and 100 mg of fungal hypha (wet weight) were harvested, and DNA was extracted using DNA extraction kit according to the manufacture protocol (ZYMO RESEARCH, California USA).

### Conserved Sequences Amplification by PCR

PCR reactions were performed using a labcycler (SensoQuest, Germany) in 25 μl PCR mixture containing 2 μl of extracted DNA (approximately 50 ng), 12.5 μl of DreamTaq green PCR master mix (Thermo scientific, Massachusetts USA), 1 μl of each primer (10 μM) and 8.5 μl PCR grade water. For bacterial isolates the 16S region was amplified by using the primers P3MOD (5′-ATTAGATACCCTDGTAGTCC-3′; SEQ ID NO: 1) and Pc5B (5′-TACCTTGTTACGACTT-3′; SEQ ID NO: 2) with the following cycling program: 95° C. for 5 min, 34 cycles at 95° C. for 60 s, 50° C. for 60 s and 72° C. for 90 s, with a final extension at 72° C. for 5 min. For fungal isolates the ITS region was amplified by using the primers ITS1 (5′-TCCGTAGGTGAACCTGCGG-3′; SEQ ID NO: 3) and ITS4 (5′-TCCTCCGCTTATTGATATGC-3′; SEQ ID NO: 4) with the following cycling program: 96° C. for 5 min, 39 cycles at 96° C. for 45 s, 55° C. for 45 s and 72° C. for 60 s, with a final extension at 72° C. for 5 min. For the identification to the species level of Bacillus spp. rpoB and recA sequences were amplified using: rpoB1206 (5′-ATCGAAACGCCTGAAGGTCCAAACAT-3′; SEQ ID NO: 5) and rpoBR3202 (5′-ACACCCTTGTTACCGTGACGACC-3′; SEQ ID NO: 6), with the following cycling program: 95° C. for 3 min, 35 cycles at 95° C. for 20 s, 55° C. for 30 s and 72° C. for 90 s, with a final extension at 72° C. for 5 min; and, recA-F (5′-GATCGTCAAGCAGCCTTAGAT-3′; SEQ ID NO: 7) and recA-R (5′-TTACCGACCATAACGCCGAC-3′; SEQ ID NO: 8), with the following cycling program: 95° C. for 5 min, 30 cycles at 95° C. for 30 s, 45° C. for 30 s and 72° C. for 60 s, with a final extension at 72° C. for 5 min, accordingly. For other gram positive bacteria, the 16S region was amplified by using the primers BSF8/20 (5′-AGAGTTGATCCTGGCTCAG-3′; SEQ ID NO: 9) and BSR1114/16 (5′-GGGTTGCGCTCGTTRC-3′; SEQ ID NO: 10) with the following cycling program: 96° C. for 5 min, 35 cycles at 95° C. for 45 s, 50° C. for 45 s and 72° C. for 60 s, with a final extension at 72° C. for 5 min. For identification to the species level of endophytic fungi, the R tubulin region was amplified by using the primers Bt1a (5′-TTCCCCCGTCTCCACTTCTTCATG-3′; SEQ ID NO: 11) and Bt1b (5′-GACGAGATCGTTCATGTTGAACTC-3′; SEQ ID NO: 12) with the following cycling program: 95° C. for 5 min, 39 cycles at 95° C. for 45 s, 54° C. for 60 s and 72° C. for 90 s, with a final extension at 72° C. for 5 min. Additionally, the EF-1α region was amplified by using the primers EF1-728F (5′-CATCGAGAAGTTCGAGAAGG-3′; SEQ ID NO: 13) and EF1-986R (5′-TACTTGAAGGAACCCTTACC-3′; SEQ ID NO: 14) with the following cycling program: 95° C. for 5 min, 39 cycles at 95° C. for 45 s, 51° C. for 45 s and 72° C. for 90 s, with a final extension at 72° C. for 5 min.

### Sequencing

PCR products (3 μl) were subjected to agarose gel electrophoresis (1.2%) and visualization under UV light, by soaking the gel in ethidium bromide solution for 3 min followed by washing in water for 5 min. PCR product (22 μl) was purified using fragment DNA purification kit (iNtRON BIOTECHNOLOGY, South Korea). The purified PCR product (30 μl of ˜20 ng/μl) was sent for sequencing (Macrogen Europe, the Netherlands). The sequencing results were compared to the National Center for Biotechnology Information (NCBI) GenBank database using the Basic Local Alignment Search Tool (BLAST) (https://blast.ncbi.nlm.nih.gov/Blast.cgi) for the identification of the bacterial/fungal species (Table 3).

Isolate ABp5 was sequenced (whole genome sequencing, NovoGene, Beijing China). Genome reads were compared to a known reference genome using gene annotations. Annotated fragments were compared to the NCBI GeneBank using BLAST.

Introduction of PU10GFP into Ulva sp.

A single colony of PU10GFP was incubated for 48 h at 25° C., 150 rpm in LB broth medium. After 48 h, the bacterial growth was washed twice with Ulva rearing water (URW) (3.7% NaCl (or 37 ppt)) as follows: centrifuging at 6,000 rpm for 10 minutes and discarding the upper phase. Then, adding URW (half volume of growth media), re-suspending the bacterial pellet. Ulva sp. reared at the Department of Plant Pathology & Weed Research, ARO the Volcani Center under controlled conditions (23° C.±2, daylight, constant air bubbling) were sampled and brought to the lab. Ulva thalli were cut into approx. 4×4 cm pieces. Cut thalli were either scratched using a thin needle or introduced to a rotated container with URW and 10×10 cm rocks, for 30 minutes, imitating the natural habitat damage vectors on the shoreline. Thalli were placed in an empty petri plate with PU10GFP solution to cover the thalli. URW only, was used in the control treatment. Thalli were then incubated for four days at room temperature. After four days, thalli were washed four times with URW and placed in a glass beaker with air bubbled URW. Introduction of endophytic PU10GFP to Ulva sp. was validated 24 hr post wash, by microscopically imaging of the Ulva thalli (FIG. 1). Image acquisition was done using a Leica SP8 laser scanning microscope (Leica, Wetzlar, Germany), equipped with a solid-state laser, HC PL APO CS2 40×/1.10 WATER objective (Leica, Wetzlar, Germany) and Leica Application Suite X software (LASX, Leica, Wetzlar, Germany). Imaging of GFP signal was done using the 488 nm laser light and the emission was detected in a range of 500-525 nm. Auto-fluorescence of the chloroplasts detected in a range of 650-700 nm. GFP intensity was measured using the ImageJ software (NIH.gov).

PU10GFP presence in Ulva sp. over time

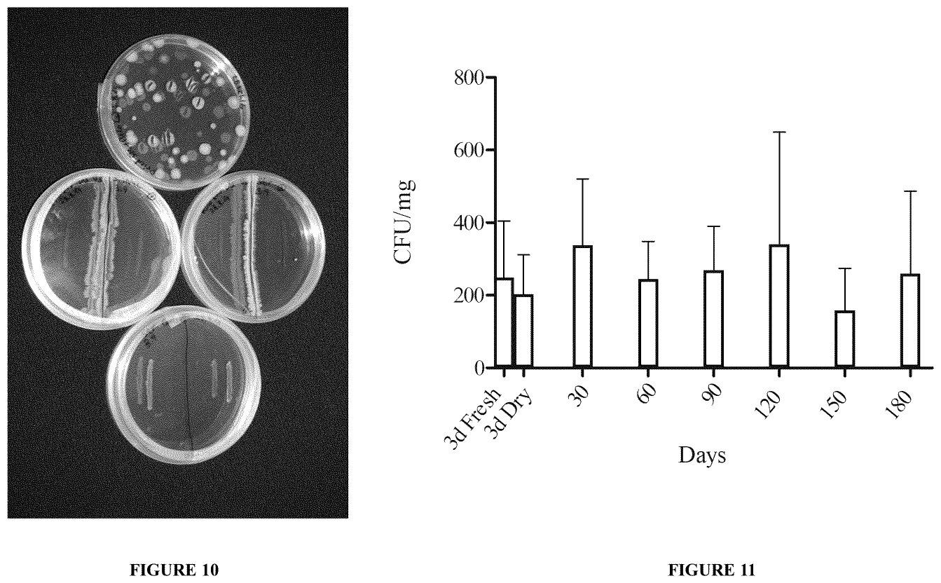

Fluorescence intensity of treated and untreated Ulva thalli was measured every 2-3 days for two weeks. In addition to fluorescence activity, bacterial CFU/sample was evaluated using antibiotic resistance (chloramphenicol) of the transformed bacteria. Ulva thalli were sampled at days 0, 4, 7, 14 and then, every 15 days over 5 months. At each sampling time, approx. 130 mm² Ulva sp. samples (in triplicates) were crushed with 500 μL sterile saline (0.9% NaCl) in a small 4×4 cm double layer nylon sleeve. Ulva sp. was cultured on LB agar plates amended with chloramphenicol (10 μg/ml) and incubated at 25° C. for 72 h followed by CFUs evaluation (FIG. 10 upper plate).

In-Vitro Bioactivity Test of PU10GFP Re-Isolated from Inoculated Ulva sp.

A single colony of PU10GFP isolated from the introduced alga, was grown on LB agar plate, as a single line in the middle of the plate (FIG. 10 middle) and incubated at 25° C. for a week. Later, the fish pathogen Streptococcus iniae was inoculated on both sides of the plate about 1 cm from the endophyte colony. Fish pathogen was inoculated on a clean LB agar plate as well, for control (FIG. 10 lower plate). The plates were incubated for another week (25° C.) and examined for pathogen growth during this week (FIG. 10).

### Presence of Active Endophytes in Freeze Dried Ulva sp.

Ulva sp. introduced with PU10GFP were reared for three days (post introduction), sampled, freeze dried (Alpha 1-4, Martin Christ, Germany) and kept in 4° C. Ulva sp. were sampled before and after freeze drying (3 days fresh and dry, respectively) and 30, 60, 90, 120 and 180 days later. Samples were crushed and the algal powder was inoculated on LB agar plates amended with chloramphenicol (10 μg/ml), incubated at 25° C. and CFUs were counted.

### Endophyte Isolation and Maintenance

The K. flava isolate (ABp5) that was cultured for use in the present study was obtained as an endophyte from the seaweed Bryopsis plumose, sampled at Ashdod shore of the Mediterranean shoreline, Israel (31° 49′03.8″N 34° 38′24.9″E). Seaweed was surface sterilized and incubated on a growth media. The endophyte grew out of the seaweed thallus and a single colony was performed to the isolate (Deutsch et al., 2021). The endophyte was stored for longer periods by freezing 0.5 ml of the isolate in LB media (Acumedia, Lansing, MI, United States) with 0.5 ml of 30% glycerol at −80° C.

### Bioactivity Assays

The K. flava isolate was spread on NPDA (½ nutrient agar+½ potato dextrose agar) (Acumedia, Lansing, MI, United States) and incubated at 25° C. for seven days. Then, the oomycote pathogen (S. parasitica) was introduced to the plate by adding a plug of PDA harboring the S. parasitica mycelia and incubated at 25° C. The activity was examined by using a one compartment petri dish, or a two-compartments petri dish (for volatile compounds). The effect of K. flava on S. parasitica was examined after 3 days, by comparing the growth of S. parasitica with that of a one in the absence of K. flava. The viability of the pathogen was evaluated by transferring inoculum plugs to fresh NPDA plates and observing the growth developed within the next 3 days. All experiments were performed in triplicates.

### Metabolite Identification

K. flava was grown on LB agar in 20 ml headspace screw top vials (Thermo scientific, Langerwehe, Germany). The isolate was spread on the LB agar in each vial and incubated at 25° C. for 3 days. A vial with only LB agar was incubated as a control, in order to subtract the media's volatiles from the sample. The vial was then introduced with SPME fiber assembly with a 50/30 m polydimethylsiloxane/divinylbenzene/carboxen (PDMS/DVB/CAR), stableflex (2 cm) 24Ga, menual holder, 3pk (Supelco, Bellefonte, PA, USA) for 24 hours. The exposed SPME fiber was then inserted into the injector port of a GC-MS apparatus for 10 min. Volatile compounds were analyzed on a GC 7890B, MSD 5977A apparatus, equipped with a HP-5MS 5% phenyl methyl silox, 1.33 m×150 μm×0.25 μm in length, diameter, and bore (Agilent Technologies, San Diego, CA, USA). The injector temperature was 160° C. and pulsed split less injection was used. The detector temperature was 280° C. The oven temperature was held at 50° C. for 2 min, then increased to 180° C. at a rate of 8° C./min, and then to 280° C. at 50° C./min. The recorded mass range was 40 to 800 m/z, with electron energy of 70 eV. The GC-MS spectrum profiles were analyzed with Mass Hunter software combined with NIST 14 library. The volatiles were identified by comparison of their retention indices with published values or with spectral data obtained with standards.

For the chemical compound 8-Nonenoic acid confirmatory identification was made by comparing the GC-MS data of endophytic products with available authentic standard, obtained from Sigma-Aldrich (St. Louis, MO, USA).

### Compound Bioassays

The inhibition activity of the compound 8-Nonenoic acid was examined in both liquid and solid media. For liquid assay, a plug of PDA harboring the S. parasitica mycelia was introduced to 1 ml of sterile double distilled water (DDW) with 10 μl of potato dextrose broth (PDB) and varying concentrations of 8-Nonenoic acid (0, 0.1, 0.5, 1, 2.5, 5, 7.5, 10, 20 μg/ml) in 48 well plate and incubated at 19° C. for 3 days. For solid assay, a plug of PDA harboring the S. parasitica mycelia was introduced to 50 mm PDA petri plates with varying concentrations of 8-Nonenoic acid (as described above) and incubated at 19° C. for 3 days. For both liquid and solid, mycelium growth radios was measured and S. parasitica plugs viability were evaluated by transferring them to fresh PDB/PDA and observing the growth developed within 3 days. All experiments were performed in triplicates.

8-Nonenoic Acid Vs. Saprolegnia parasitica in Tilapia Eggs Bioassay Test

Tilapia (Oreochromis mossambicus) eggs, 3 days post fertilization, were collected from the hatchery at the department of fisheries in ARO the Volcani Center and brought to the lab. Eggs were rinsed with aquarium water. Eggs were introduced to 100 ml flasks (25 ml water in each flask), 6 eggs for each flask. Eggs were introduced with Saprolegnia parasitica plugs in the flask or with Saprolegnia parasitica plugs and 10 μg/ml 8-Nonenoic acid. Control eggs were not treated and/or supplemented. Flasks were incubated on a shaker under controlled conditions: 26° C., 95 rpm, and day light for a week.

### Example 1

### Macro-Algae Sampling

Twenty macro-algae were collected from different locations in Israel. Twelve different genera were sampled from different marine and freshwater sources and locations. The algal genus and site locations are presented in Table 1.

TABLE 1
Algae's isolated endophytes from different water sources in Israel, with the
percentage of bioactive isolates

| | | | Number of | | | % of |
|---|---|---|---|---|---|---|
| Collection | Habit | | Isolates | | *Bioactive | Bioactive |
| Site | at | Algae | Bacterial | Fungal | Isolates | Isolates |
| Herzliya | Marine | Ulva sp. | 12 | — | 3 | 25 |
| Beach | | Gracilaria sp. | 24 | 8 | 10 | 31 |
| 32°09'28.1″ | | | | | | |
| N | | | | | | |
| 34º47'41.0″ | | | | | | |
| E | | | | | | |
| Tel Aviv | Marine | Alsidium | 5 | 2 | 3 | 43 |
| Beach | | corallinum | | | | |
| 32°08'39.6″ | | Acanthophore | 3 | 2 | 3 | 60 |
| N | | najadiformis | | | | |
| 34°47'27.7″ | | Padina | 7 | 4 | 4 | 36 |
| E | | pavonica | | | | |
| | | Enteromorpha | 9 | 1 | 2 | 20 |
| | | ralfsii | | | | |
| Palmachim | Marine | Ulva sp. | 11 | 1 | 2 | 17 |
| Beach | | Jania rubens | 9 | 2 | 5 | 45 |
| 31º55'47.5″ | | Spyridia | 9 | 1 | 5 | 50 |
| N | | Harvey | | | | |
| 34º41'53.7″ | | Alsidium | 3 | — | 2 | 67 |
| E | | corallinum | | | | |
| | | Laurencia | 6 | — | 5 | 83 |
| | | papillosa | | | | |
| | | Acanthophore | 4 | 2 | 1 | 17 |
| | | najadiformis | | | | |
| | | Padin | 9 | 1 | 6 | 60 |
| | | pavonica | | | | |
| Ashdod | Marine | Ulva sp. | 2 | 2 | 2 | 50 |
| Beach | | Bryopsis | 5 | 2 | 5 | 71 |
| 31º49'03.8″ | | plumosa | | | | |
| N | | Enteromorpha | 2 | — | 0 | 0 |
| 34º38'24.9″ | | compressa | | | | |
| E | | | | | | |
| En Sappir | Fresh | Pithophora sp. | 4 | — | 2 | 50 |
| spring | water | | | | | |
| 31º45'39.1″ | | | | | | |
| N | | | | | | |
| 35°07'53.0″ | | | | | | |
| E | | | | | | |
| Sea of | Fresh | Pithophora sp. | 7 | -— | 3 | 43 |
| Galilee | water | | | | | |
| 32°42'25.3″ | | | | | | |
| N | | | | | | |
| 35°35'43.5″ | | | | | | |
| E | | | | | | |
| Sea of | Fresh | Pithophora sp. | 1 | 1 | 0 | 0 |
| Galilee | water | | | | | |
| (Unknown | | | | | | |
| spring) | | | | | | |
| 32°42'24.7″ | | | | | | |
| N | | | | | | |
| 35°35'43.0″ | | | | | | |
| E | | | | | | |
| Hamat | Thermo- | Leptolingbya | 10 | 2 | 1 | 8 |
| Gader | Mineral | sp. | | | | |
| Mineral | water | | | | | |
| springs | | | | | | |
| 32°40'56.1″ | | | | | | |
| N | | | | | | |
| 35º39'59.2″ | | | | | | |
| E | | | | | | |

*″Bioactive Isolates″ is defined as an endophyte that inhibits at least one of the aquaculture pathogens in in vitro bioactivity assays. The percentage of the bioactive isolates was calculated as the number of bioactive endophytes out of all endophytes isolated from a certain alga collected.

### Example 2

### Isolation of Endophytes

The inventors isolated 31 fungal and 142 bacterial endophytes from the twenty algae collected. Most of the macro-algae were collected from the Mediterranean shoreline (Herzliya, Tel Aviv, Palmachim, and Ashdod). The highest numbers of endophytes (148) were isolated from this environment. When comparing the number of bacterial endophytes isolated from the different algae, the inventors saw that Gracilaria sp. produced the highest number of bacterial endophytes (24) under the herein disclosed isolation method, followed by Ulva sp., and the Thermo-Mineral water cyanobacterium Leptolingbya sp. (10) (Table 1). When examining the number of isolated bacterial endophytes of the same type of algae from the different locations the inventors observed variations depending on the location of collection. For example, Ulva sp. produced similar number of bacterial endophytes from Herzliya and Palmachim areas (11 and 12 respectively) but only 2 from Ashdod location. In general, the Ashdod location was poorer in endophytes than the other locations sampled in the Mediterranean coast. The same was found for the only alga collected in fresh water. The inventors isolated more bacterial endophytes from Pithophora sp. collected at the Sea of Galilee Lake (7) than from Pithophora sp. collected in a spring running into the lake (1). The number of fungal isolates was much lower than bacteria in all algae at all locations.

### Example 3

### Bioactivity Assays

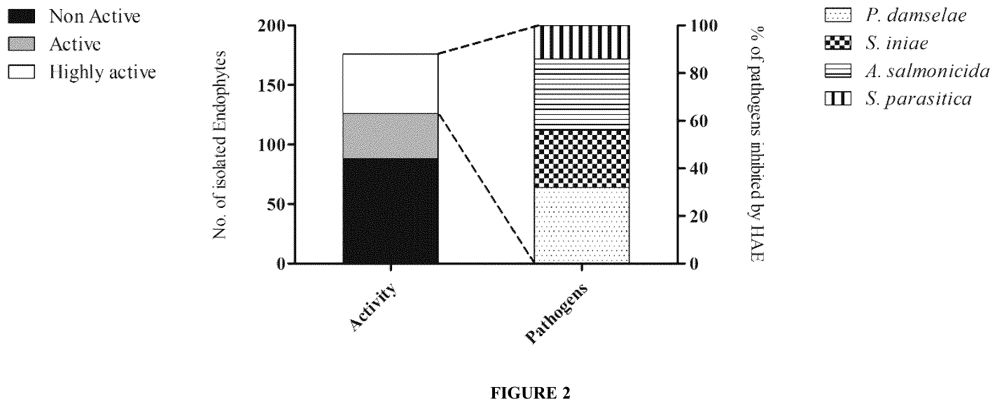

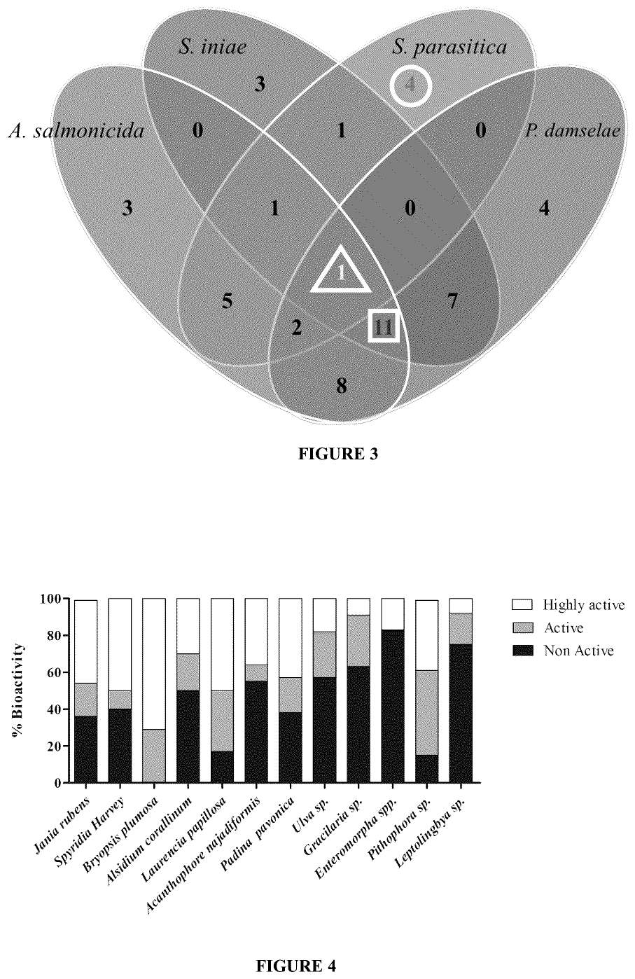

All 173 isolates were tested for their ability to inhibit the four-aquaculture pathogens in in vitro assays (Tables 1, and 2). Fifty endophytes (1 fungal and 49 bacterial) were found to have strong activity (pathogen inhibition and loss of viability) against at least one of the four pathogens tested. The inventors designated the activity of the tested endophytes to three categories: Highly Active Endophytes (HAE—50 isolates) as described above; Active Endophytes (AE—38 isolates) if they had some inhibition abilities, or as Non-Active Endophytes (NAE—88 isolates) if they had no bioactivity at all (FIG. 2). Only one endophyte (AJr10) was highly active against all four pathogens while 11 were found to be active against all the bacterial pathogens. Four endophytes were active specifically against the oomycete (ABp5, FWTA4, AU4 and TAEnN2) (FIG. 3 and Table 2).

The inventors further analyzed the herein described findings regarding the endophytes' bioactivity, with reference to the sampled alga, endophytes activity against pathogens, and sampling sites. To analyze the distribution of active endophytes in the different algae collected the inventors normalized the number of bioactive isolates from each algal genus to percentage (FIG. 4). The average HAE for all the sampled algae was found to be 35%. While the highest percent of HAE (71%) was isolated from B. plumosa (seawater) and the lowest is (8%) from Leptolingbya sp. (thermo-mineral water). The highest percent of NAE (83%) was isolated from Enteromorpha spp. (seawater), while B. plumosa had no NAE (FIG. 4 and Table 1). At the phylum level; algae from the phyla Rhodophyta had the highest percent of active endophytes (8 sampled algae 49.5% active isolates), while the phyla Chlrophyta had 9 sampled algae with 30.6% active isolates. The alga Padina pavonica of the phyla Ochrophyta were sampled twice and had 48% active isolates. The only Cyanobacteria sampled was Leptolingbya sp. and had the lowest (8%) active isolates (Table 1).

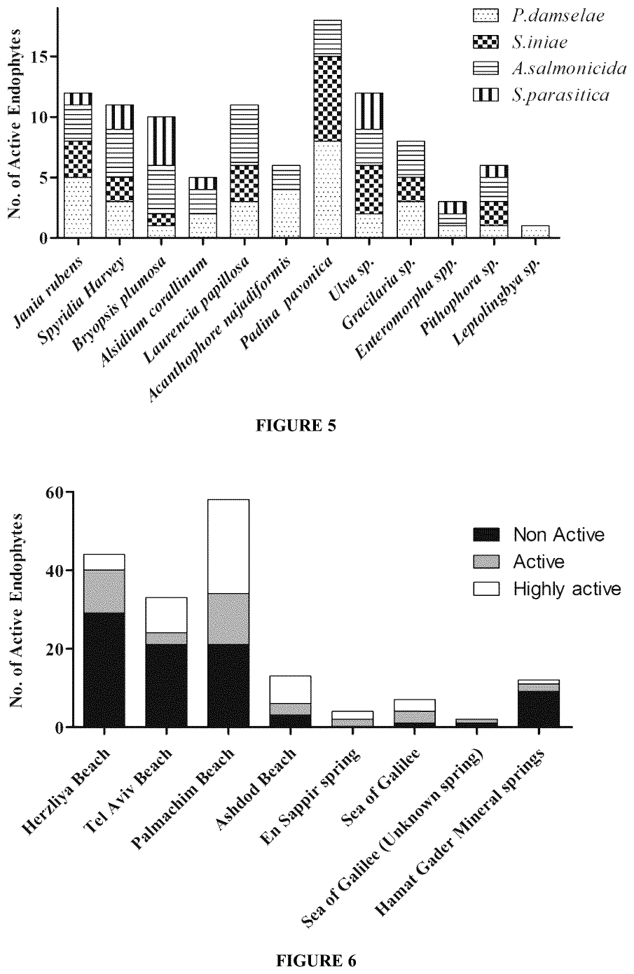

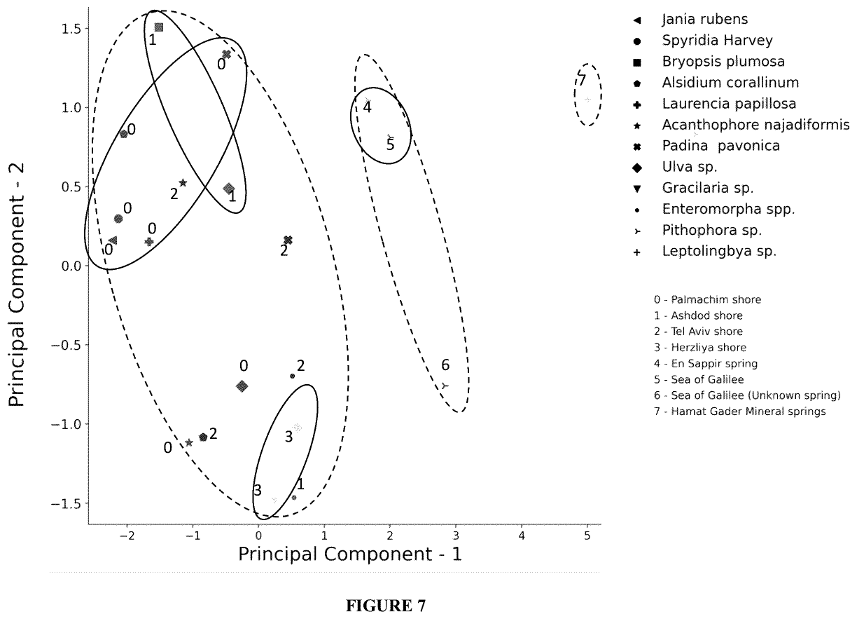

As described, the inventors used four pathogens to test the bioactivity of the endophytes isolated from the different algae. These pathogens represent some of the most influencing disease agents in aquaculture and in fish farming in specific. The four pathogens, three bacteria and one oomycote are: Photobacterium damselae, Streptococcus iniae, Aeromonas salmonicida and Saprolegnia parasitica (respectively). P. damselae was the most susceptible pathogen to the endophytes the inventors tested in this study (FIG. 2). Thirty-three endophytes from the different algae inhibited and caused mortality of P. damselae (FIG. 3). Only two out of the six HAE (33%) from the freshwater algae Pithophora spp. and Leptolingbya sp. were found active against P. damselae, while 32 of the 44 HAE (73%) from seaweeds were active against this pathogen. The highest numbers of active endophytes against the bacterial pathogens were isolated from Padina pavonica (FIG. 5). Yet, although these endophytes inhibited all four pathogens, none was active enough to induce mortality of the pathogenic oomycote S. parasitica in the viability tests (Table 2). A. salmonicida was the second most susceptible pathogen to the active endophytes. The only alga from which no isolates were active against A. salmonicida is the cyanobacterium Leptolingbya sp. (FIG. 5). Only one fungal endophyte displaying bioactivity with mortality of the pathogens was isolated. This endophyte (AU4) from Ulva sp. has the ability to inhibit, not cause mortality of S. iniae (Weak inhibition) and A. salmonicida (Moderate inhibition), and while having strong activity (85% inhibition) with inducing mortality of the oomycetes S. parasitica (Table 2 and FIG. 5).

Although algal sampling from the Mediterranean shoreline of Israel was done from locations relatively close to each other (45 km at most, from Herzliya to Ashdod), the diversity of algae genus, the number of endophytes isolated from the different algae and the activity of these endophytes was high. It maybe that the geographic distance has no real influence and differences in habitat as abiotic parameters (nutrients, temperature, salinity, pollution etc.), are the factors influencing the endophytic diversity in the algae. Nevertheless, the inventors found that the number of isolates from Ashdod shoreline was somewhat low (13), yet with high percentage of HAE (54%). Herzliya, Tel Aviv and Palmachim shorelines produced higher numbers of endophytes with lower percentage of active isolates (29.5, 36 and 45%, respectively) (Table 1).

Alga collected from Hamat Gader site (thermo-mineral water) had a high number of isolates but low number of active endophytes. Interestingly, isolates from the Sea of Galilee showed higher bioactivity (HAE) compared to isolates from a spring that flows into the Sea of Galilee (no HAE), although they were isolated from the same algal genus (FIG. 6 and Table 1).

TABLE 2
Highly active endophytes from different algae, sorted by their ability to inhibit
and/or kill four major aquaculture pathogens

| | | Pathogens | | | | | | | |
|---|---|---|---|---|---|---|---|---|---|
| | | | | | | | | **S. parasitica | |
| Algae | | *P. damselae | | *S. iniae | | *A. salmonicida | | % | |
| Host | Isolate | Inhibition | Lethality | Inhibition | Lethality | Inhibition | Lethality | Inhibition | Lethality |
| Jania | AJr3 | +++ | + | +++ | + | ++ | + | 45 | − |
| rubens | AJr7 | +++ | + | − | − | + | − | 39 | − |
| | AJr9 | +++ | + | +++ | − | +++ | + | 89 | + |
| | AJr10 | +++ | + | +++ | + | ++ | + | 63 | + |
| | AJr15 | +++ | + | +++ | + | − | − | 37 | − |
| Spyridia | PSH1 | ++ | − | +++ | + | + | − | 61 | − |
| Harvey | PSH3 | +++ | + | + | − | ++ | + | 20 | − |
| | PSH5 | ++ | + | +++ | + | ++ | + | 28 | − |
| | PSH6 | +++ | + | +++ | − | ++ | + | 100 | + |
| | PSH7 | − | − | − | − | ++ | + | 100 | + |
| Bryopsis | ABp1 | ++ | − | + | − | +++ | + | 100 | + |
| plumosa | ABp2 | +++ | + | +++ | + | + | + | 69 | − |
| | ABp3 | +++ | − | − | − | +++ | + | 100 | + |
| | ABp4 | +++ | − | − | − | +++ | + | 100 | + |
| | ABp5 | − | − | − | − | − | − | 100 | + |
| Alsidium | PAc1 | +++ | − | + | − | +++ | + | 100 | + |
| corallinum | PAc2 | ++ | − | +++ | − | +++ | + | 60 | − |
| | TAAN 4 | +++ | + | + | + | + | − | 7 | − |
| Laurencia | PLp2 | +++ | + | +++ | + | ++ | + | 37 | − |
| papillosa | PLp5 | +++ | + | +++ | + | ++ | + | 20 | − |
| | PLp6 | +++ | + | +++ | + | ++ | + | 58 | − |
| Acanthophore | PAn4 | +++ | + | + | − | ++ | + | 0 | − |
| najadiformis | TAAnN 1 | +++ | + | + | − | + | + | 0 | − |
| | TAAnN 2 | +++ | + | + | − | +++ | + | 0 | − |
| | TAAnN 3 | +++ | + | + | − | + | + | 0 | − |
| Padina | PP1 | +++ | + | +++ | + | − | − | 0 | − |
| pavonica | PP2 | +++ | + | +++ | + | ++ | + | 48 | − |
| | PP3 | +++ | + | +++ | + | ++ | − | 0 | − |
| | PP6 | +++ | + | +++ | + | ++ | + | 60 | − |
| | PP7 | ++ | − | +++ | + | + | − | 40 | − |
| | PP8 | +++ | + | +++ | + | − | − | 0 | − |
| | TAPaN 1 | +++ | + | + | − | + | + | 0 | − |
| | TAPaN 2 | +++ | + | + | − | + | + | 0 | − |
| | TAPaN 3 | +++ | + | ++ | + | +++ | − | 13 | − |
| Pithophora | KMFWTA1 | + | − | +++ | + | +++ | − | 36 | − |
| spp. | KMFWTA3 | +++ | + | +++ | + | +++ | − | 57 | − |
| | KMFWTA4 | − | − | − | − | − | − | 100 | + |
| | FWTa1 | − | − | − | − | +++ | + | 0 | − |
| | FWTa5 | − | − | + | − | +++ | + | 0 | − |
| Leptolingbya sp. | HGCy9 | +++ | + | − | − | + | − | 0 | − |
| Ulva sp. | PU9 | ++ | − | +++ | + | − | − | 100 | + |
| | PU10 | ++ | − | +++ | + | +++ | + | 100 | + |
| | AU1 | +++ | + | ++ | − | ++ | − | 5 | − |
| | AU4*** | − | − | + | − | ++ | − | 85 | + |
| | HU4N5 | +++ | + | ++ | + | +++ | + | 0 | − |
| Gracilaria | HG2N7 | +++ | + | + | − | ++ | + | 0 | − |
| sp. | HG3N2 | +++ | + | ++ | + | ++ | + | 0 | − |
| | HG3M1 | +++ | + | ++ | + | + | + | 67 | − |
| Enteromorpharalfsii | TAEnN2 | + | − | − | − | + | − | 100 | + |
| | TAEnN4 | +++ | + | − | − | ++ | − | 0 | − |

*Challenged bacterial pathogens' (P. damselae, S. iniae, A. salmonicida) growth is estimated qualitatively using four levels of inhibition as compared to control: 1. Very strong inhibition-no pathogen's growth (+++), 2. Moderate inhibition- low pathogen's growth (++), 3. Weak inhibition-pathogen's growth slightly lower than of the control (+) and 4. No inhibition- pathogen's growth equal to control (−).
**Challenged Oomycete pathogen's (S. parasitica) growth is estimated quantitatively by the percentage of inhibition compared to control, measured as the radius of the pathogenic growth in mm: Very strong inhibition, no pathogenic growth (100). No inhibition, full pathogenic growth (0).
Pathogens' lethality as tested in viability assays: Pathogen was killed by the endophyte (+).
Pathogen was not killed by the endophyte (−) .*** A fungal endophyte.

### Example 4

### Endophytes Molecular Identification

Of the most active endophytes, the inventors identified 23 to the genus level using ribosomal DNA conserved sequences: 16S for bacteria, and ITS 5.8S for fungi. Six endophytes were identified to the species level using the conserved sequences rpoB, recA, and gyrB for bacterial endophytes, and EF-1α and β tubulin for fungal endophytes. The molecular identification of the endophytes revealed that the majority of the active endophytes isolated from the different algae belong to the Bacillus spp. The isolate ABp5 of Bryopsis plumosa from Ashdod shoreline was identified as Kocuria flava, HG9 of Leptolingbya sp. from the thermal springs in Hamat Gader was identified as Lysinibacillus sp., TAEnN2 isolated from Enteromorpha ralfsii at Tel Aviv shoreline was identified as Staphylococcus warneri and the fungal isolate AU4 from Ulva sp. collected at Ashdod was identified as Beauveria sp. (Table 3).

TABLE 3
Identification of the highly active endophytic bacteria and fungi

| Isolate | Specie | | | Accession | Final specie |
|---|---|---|---|---|---|
| name | 16S | rpoB | recA | numbers | identification |
| AJr9 | B. | Bacillus | Bacillus | MT177330 (16S), | Bacillus |
| | amyloliquefaciens | subtilis | subtilis | MT326591 (rpoB), | subtilis |
| | | | | MT219829 (recA) | |
| ABp5 | Kocuria | Kocuria flava | Kocuria | MT183699 (16S) | Kocuria flava |
| | kristinae | | flava | MT901192 (rpoB) | |
| | | | | MT901193 (recA) | |
| PU9 | B. velezensis | B. velezensis | B. | MT178235 (16S), | Bacillus |
| | | | velezensis | MT326592 (rpoB), | velezensis |
| | | | | MT219830 (recA) | |
| PU10 | Bacillus | B. velezensis | Bacillus | MN134034 (16S), | Bacillus |
| | subtilis | | subtilis | MN306258 (rpoB), | subtilis |
| | | | | MT123513 (recA) | |
| HU4N5 | B. safensis | B. safensis* | B. safensis | MT232982(16S), | Bacillus |
| | | | | MT465311 (gyrB), | safensis |
| | | | | MT219831 (recA) | |
| AU4 | Parengyodontium | Beauveria | Beauveria | MT180744 (ITS | Beauveria sp. |
| | album | bassiana | hoplocheli | 5.8S), | |
| | | | | MT326590 (Btla/b), | |
| | | | | MT326593 (EF1) | |
| AJr10 | B. safensis | | | MT180808 (16S) | |
| PSH6 | B. velezensis | | | MT187620 (16S) | |
| PSH7 | Bacillus | | | MT192301 (16S) | |
| | subtilis | | | | |
| ABp4 | Bacillus | | | MT187638 (16S) | |
| | subtilis | | | | |
| PLp5 | B. safensis | | | MT186284 (16S) | |
| PP2 | B. pumilus | | | MT116792 (16S) | |
| PP6 | B. pumilus | | | MT186599 (16S) | |
| KM3 | B. pumilus | | | MT186687 (16S) | |
| KM4 | Pseudomonas | | | MT186697 (16S) | |
| | alcaligenes | | | | |
| PAn4 | B. | | | MT192227 (16S) | |
| | megaterium | | | | |
| PAc1 | Bacillus | | | MT188131 (16S) | |
| | subtilis | | | | |
| HG9 | Lysinibacillus | | | MT188141 (16S) | |
| | sp. | | | | |
| HG3M1 | Bacillus | | | MT242588 (16S) | |
| | subtilis | | | | |
| HG3N2 | B. pumilus | | | MT252929 (16S) | |
| TAPaN3 | B. cereus | | | MT256171 (16S) | |
| TAEnN2 | Staphylococcus | | | MT272978 (16S) | |
| | haemolyticus | | | | |
| TAAnN2 | B. altitudinis | | | MT322994 (16S) | |

*The isolate HU4N5 was identified as Bacillus safensis using the conserved gene of gyrB.
** The isolate AU4 was identified as Parengyodontium album, Beauveria bassiana and Beauveria hoplocheli using the conserved genes ITS 5.8S, Btla/b and EF1, respectively.
The presented identifications correlate the highest score of all identifications, given by NCBI GenBank database using BLAST.

### Example 5

### Preparation of Probiotic Algae (Ulva sp.) Powder as Food Additive

The active endophyte (genus: Bacillus) is inoculated in 50 ml LB solution at 25° C., (rotated at 150 RPM) for 48 hours. After 48 h, the bacterial culture is collected by centrifugation at 6,000 RPM for 10 min, after which the supernatant is discarded. Then, the pellet is washed with artificial seawater (˜37% _{∞} salinity) by centrifugation at 6,000 RPM for 10 min, after which the supernatant is discarded. Twenty-five (25) ml of artificial seawater are added, and pellet is re-suspended.

Ulva sp. is harvested, and Ulva thallus are wounded by scratching the algae surface.

The endophyte suspension is mixed with the treated algae and placed in a partially opened container at room temperature for four days. After four days, the algae are washed to remove the excess endophyte solution with artificial seawater, four times and incubated in fresh seawater with continuous aeration.

Three (3) days later, the algae is freeze-dried, overnight, in a Lyophilizer. The dried alga is ground into a powder and stored (can be stored for at least six months). The powder can be mixed with fish feed by using gelatin, oil, or agar.

### Example 6

### Administration of Endophytes and their Metabolites

Algae powder—containing the endophytes in an endospore structure. When powder is introduced to water system, endophytes can reactivate themselves and thrive. Algae powder is mixed with fish feed by using gelatin, oil, or agar. Other methods for using the endophytes as probiotic supplements is by using a protocol for creating bacterial endospores and spray-coating or mixing them with fish feed, or by probiotic enriched Artemia as live carriers of the endophytes to the fish.

Fresh live algae—growing in the system as natural bio-filter; Ulva sp. introduced with active endophytes is added to an aquatic system with suitable growing conditions for Ulva (e.g., salinity at 37%). The endophytes in the algae secrete the active metabolites to the water, helping in preventing diseases inoculant development in the system.

Active endophytes are encapsulated and introduced as part of the bio-filter array in the aquatic systems. In this case, endophytes are confined in the capsules, however, nutrients and secreted metabolites pass through the capsule wall into the water, thereby reducing or preventing diseases inoculant development in the system.

A crude extraction of the secondary metabolites secreted to growth media after fermentation with an active endophyte, is dissolved in oil and mixed with fish feed.

Single active metabolite isolated and characterized from an active crude extraction is added to the water as a disinfectant or mixed with fish feed.

### Example 7

### Endophyte-Comprising Algae as a Feed Additive for Fish

An endophyte isolate that is found to be active against a pathogen of fish (based on laboratory tests) is introduced to Ulva sp. algae. Seven days following the introduction, the algae comprising the endophyte is dried by lyophilization. The dried alga is ground and mixed (at a known weight) with the fish feed by using gelatin or a comparable inert oil. A control alga, devoid of or not comprising the endophyte, serves as a control (e.g., for inspection and for the purpose of testing the effect of the algae only on sick and healthy fish) and undergoes a similar process. Fish, e.g., Tilapia, are infected with the pathogenic bacteria Streptococcus iniae, whereas healthy (uninfected) fish serve as a control. Each of the experimental tanks is populated with ten fish, with each treatment being assigned to three tanks. Fish are fed regularly in all treatments; wherein healthy control fish are being fed on standard feed (“Regular feed”).

### Treatments

All treatment described herein below are further summarized in Table 4.

Treatments 1 and 2—Standard feed is provided to healthy fish (negative control for the herein described tested feed; Treatment) and to sick fish (positive control for the herein described tested feed; Treatment 2).

Treatment 3—To check whether an intact/normal alga (without endophyte) affects the fish in any way (e.g., toxic to or harms them), treatment 3 is performed in which healthy fish are fed on feed mixed with algae powder that was used as a negative control group when the endophytic population introduction was performed and does not include endophytes.

Treatment 4—To check whether the algae provides a beneficiary effect to sick fish, the control algae is added to the feed provided to the sick fish.

Treatment 5—The algae powder populated with the active endophyte is added to the feed and provided to healthy fish for toxicity testing.

Treatment 6—The algae powder populated with the active endophyte is added to the feed and provided to sick fish, in order to test the efficacy of the “product” and its activity against the pathogen.

The experiment spans over a period of about a month, during which fish conditions are examined, including the state of the disease and fish survival. At the end of the experiment, the size, weight and vitality of the healthy fish (Treatments 1, 3, and 5, Table 4) are recorded in order to conclude on the adverse effects resulting from the herein disclosed probiotic product and use of same.

TABLE 4
Study design

| | | | Control | Populated | | |
|---|---|---|---|---|---|---|
| | | | algae | algae | | |
| | | | Served as | Algae | | Sick |
| | | | negative | populated | | fish |
| | | | control for | with | | Fish |
| | Name of | | endophyte | endophytes | | infected |
| | treatment | Feed | population | having anti | | with a |
| | 10 fish per | Standard | (did not | pathogenic | Healthy | pathogen |
| | tank, 3 | feed | undergo | activity | fish | susceptible |
| Treatment | tanks per | (routinely | endophyte | related to | Healthy | to the |
| # | treatment | supplemented) | introduction) | fish | fish | endophyte |
| 1 | Feed | + | | | + | − |
| | negative | | | | | |
| | control | | | | | |
| 2 | Feed | + | | | − | + |
| | positive | | | | | |
| | control | | | | | |
| 3 | Algae | + | Pre- | | + | − |
| | toxicity | | determined | | | |
| | | | weight of | | | |
| | | | algae | | | |
| | | | powder | | | |
| | | | admixed | | | |
| | | | to feed | | | |
| | | | (e.g., with | | | |
| | | | gelatin, | | | |
| | | | or oil) | | | |
| 4 | Algae | + | Pre- | | − | + |
| | positive | | determined | | | |
| | control | | weight of | | | |
| | | | algae | | | |
| | | | powder | | | |
| | | | admixed to | | | |
| | | | feed (e.g., | | | |
| | | | with | | | |
| | | | gelatin, | | | |
| | | | or oil) | | | |
| 5 | Probiotic | + | − | Pre- | + | − |
| | product of | | | determined | | |
| | the | | | weight of | | |
| | invention- | | | algae | | |
| | toxicity | | | populated | | |
| | | | | with | | |
| | | | | endophyte | | |
| | | | | powder as | | |
| | | | | admixed | | |
| | | | | to feed | | |
| 6 | Probiotic | + | − | Pre- | − | + |
| | product of | | | determined | | |
| | the | | | weight of | | |
| | invention- | | | algae | | |
| | activity | | | populated | | |
| | | | | with | | |
| | | | | endophyte | | |
| | | | | powder as | | |
| | | | | admixed to | | |
| | | | | feed | | |

### Example 8

### Introduction of PU10GFP into Ulva sp

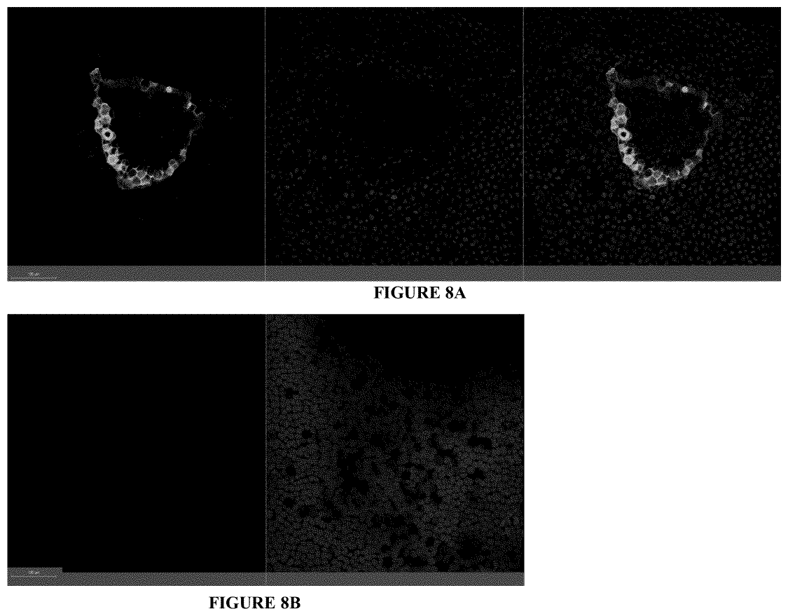

PU10GFP introduction into Ulva sp. was demonstrated using a fluorescence microscope. FIG. 8A displays the inhabitation of the edges of wounds made to the algae tissue by a cork borer. GFP was observed in the intercellular spaces of the algae (FIG. 8A), while a control Ulva sp. tissue showed no GFP emission (FIG. 81B).

### Example 9

### PU10GFP Presence in Ulva sp. Over Time

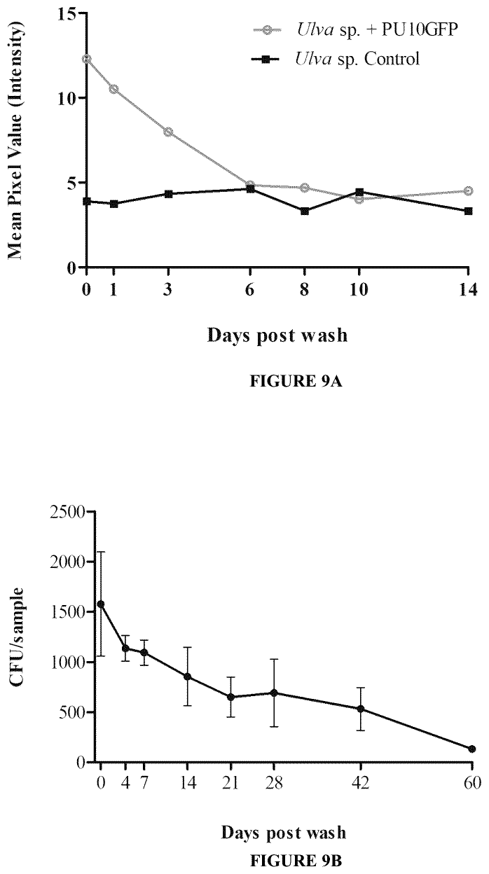

The fluorescence intensity of the introduced endophytes (PU10GFP) at the first days post wash was up to three times greater than the control (12.28 and 3.89 mean pixel values, respectively). Seven days post wash, the fluorescence intensity declined to equal with the auto-fluorescence of the control (Ulva with no introduced endophytes) (FIG. 9A). PU10GFP enquired alongside the GFP gene, an antibiotic resistance (chloramphenicol) gene. This resistance enabled the inventors to use this selection marker for PU10GFP isolation on selective media during the 60 days of the experiment. The presence of the endophytes was measured as colony forming units (CFU) per Ulva sp. sample (approx. 130 mm²) (FIG. 9B). At the first day (wash from inoculation), the amount of CFU was higher than 1,500 per sample. One week post wash, 1,000 CFU/sample of PU10GFP were isolated on the selective media. By day 21 post inoculation, the number of colonies isolated from the introduced algae decrease to 650 CFU/sample. By day 60 post inoculation, the number of colonies isolated from the introduced algae decrease to 130 CFU/sample (FIG. 9B). The inventors have results, showing PU10GFP isolated colonies in Ulva sp. until day 150 post inoculation, when the number of colonies decrease to 30 CFU/sample.

### Example 10

### In-Vitro Bioactivity Test of PU10GFP Re-Isolated from Inoculated Ulva sp

PU10GFP isolated from the introduced algae was tested against the fish pathogen S. iniae. The bioassay was performed in order to study the potential bioactivity of the endophyte over time in the algae. The bioassay was performed on endophytes from both live fresh Ulva and freeze-dried inoculated Ulva. The endophyte from both the fresh and the dried alga was active against S. iniae at the same strength as a colony of PU10GFP from a culture kept in the freezer. The pathogen did not grow on the plates and was not viable post exposer to the endophyte from all treatments (data not shown).

### Example 11

### Presence of Active Endophytes in Freeze Dried Ulva sp

PU10GFP presence and potential bioactivity in the introduced freeze-dried algae over time was evaluated by isolation of the bacteria on selective LB media (with chloramphenicol). At each time point over at least 180 days, bacterial number (CFU/mg) and ability to inhibit and kill S. iniae was recorded. The inventors found that the number of PU10GFP was stable and equal to the numbers recorded for both fresh and dry algae 3 days post wash (FIG. 11). The inventors continued one experiment for more than a year and found to have the presence of the endophytes at these numbers even after 12 months of storage. Moreover, the colonies isolated after 12 months in storage were biologically active against the pathogen (data not shown). This result supports the possibility that freeze dried Ulva can be stored for at least a year without losing PU10 as an active endophyte, when kept under the right conditions.

### Example 12

### Bioactivity Assays

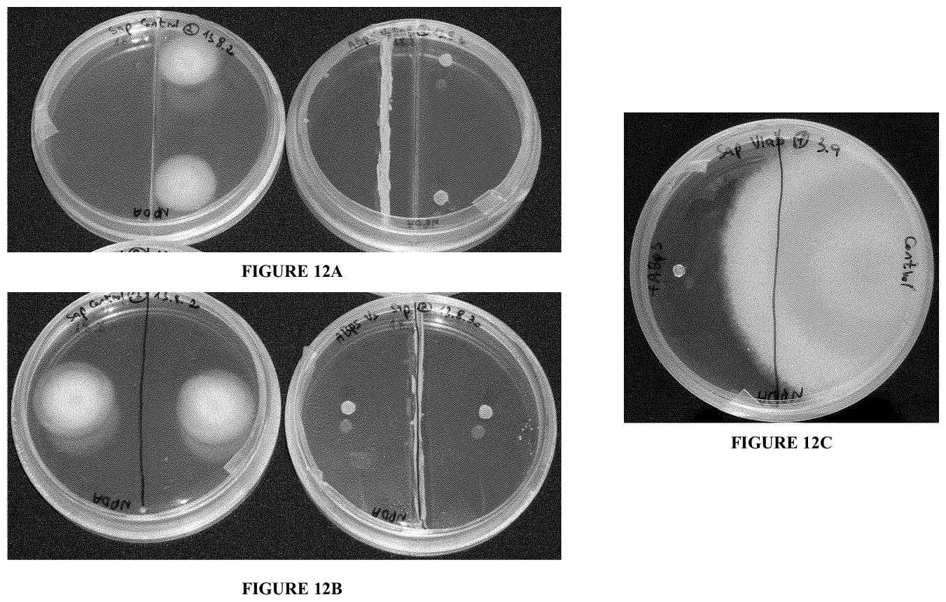

K. flava showed very strong inhibition levels against the oomycote pathogen S. parasitica. The first bioassay was of one compartment petri dish, showing full inhibition of S. parasitica compared to control plates in the absence of the endophyte (FIG. 12B). Since activity was lost in the liquid crude (when endophyte's culture was extracted for secondary metabolites), an assay for volatile activity of the endophyte was performed and a two compartments petri dish was used. The partition separated between the endophyte's agar and the pathogen's agar, thus allowing the only the endophyte's volatiles to fully inhibit the pathogen, compared to control plates wherein the endophyte was absent (FIG. 12A). S. parasitica plugs from both one and two compartments petri dish assays were tested for viability showing loss of viability in both cases compere to control (FIG. 12C).

### Example 13

### Metabolite Identification

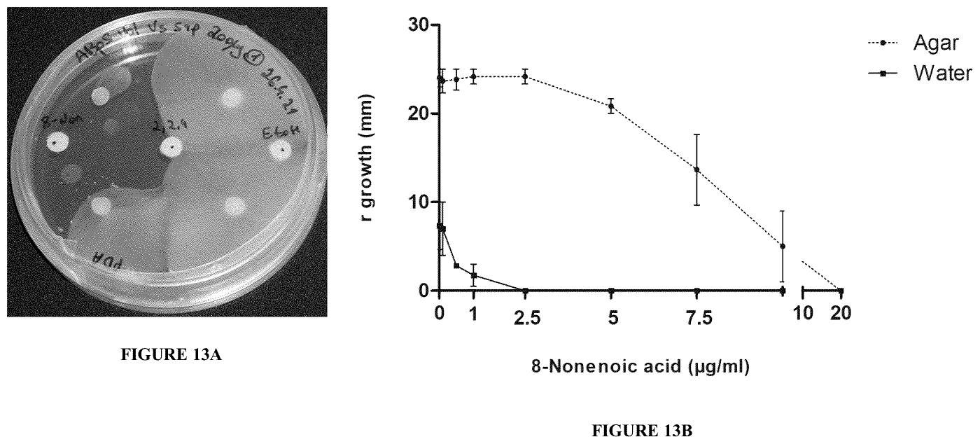

In order to further understand the basis of the bioactivity of K. flava, the inventors chemically analyzed the gas phase of the endophyte grown on LB with a GC/MS apparatus. The inventors identified 10 different compounds that could be divided among several families of chemical substances: ketones, alcohols, phenol, fatty acid, and ester. Nine out of the ten compounds identified by the GC/MS analysis, were commercially available (Oxime-, methoxy-phenyl- was not available). The inventors purchased all nine compounds and examined their ability to control the growth of S. parasitica; The inventors found one compound (8-Nonenoic acid) to have strong ability to inhibit S. parasitica growth (FIG. 13A). Final identification for the active compound was based on comparison with authentic standard. The standard yielded retention time and mass spectra that was identical to the endophytic product.

### Example 14

### Compound Bioassays

In order to find the best inhibition ability for the compound 8-Nonenoic acid, a concentration assay of two states (liquid and solid) was performed. The mycelium growth radios of S. parasitica in water was smaller than in agar (10 and 23 mm, respectively) when no compound was added (control). No inhibition in S. parasitica growth was recorded when adding a concentration of 0.1 g/ml in both water and agar. When the applied concentration of the compound was increased in water (0.5-2.5 μg/ml), the growth radios decreased rapidly to a level of full inhibition at 2.5 μg/ml, while no inhibition was recorded in the agar phase at these concentrations. Only when applying a concentration of 5 μg/ml or greater in agar, the growth radios decreased, up to a level of full inhibition at 20 μg/ml (FIG. 13B). The fully inhibited S. parasitica plugs that were transferred to a new plate for viability assays did not grow, thus showing complete loss of viability of the pathogen when fully inhibited.

### Example 15

### 8-Nonenoic Acid Vs. Saprolegnia parasitica in Tilapia Eggs Bioassay Test

Tilapia eggs were introduced with S. parasitica plugs (as negative control) or with S. parasitica plugs and 10 μg/ml 8-Nonenoic acid, as described above.

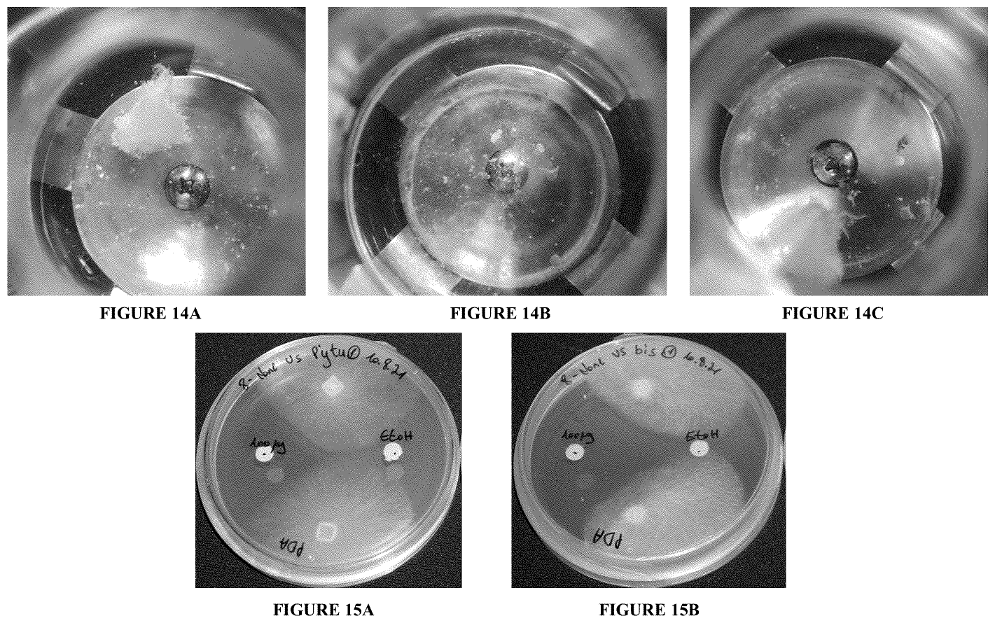

After one week, survived eggs were hatched. Eggs incubated with S. parasitica plugs did not survive (FIG. 14A), whereas positive control eggs did survive (FIG. 14B; intact un-treated eggs). Eggs that were incubated with both S. parasitica plugs and 10 μg/ml 8-Nonenoic acid did survive (FIG. 14C).

### Example 16

### In-Vitro Bioactivity Assays of 8-Nonenoic Acid Against Other Pathogens

Since 8-Nonenoic acid was found to be active against the oomycote S. parasitica, further bioassays were performed against oomycete pathogens. One hundred (100) μg of 8-Nonenoic acid were added on a paper disk and introduced to PDA petri plates together with Pythium aphanidermatum plugs (FIG. 15A) and showed inhibition of the oomycote mycelium. A similar assay was performed on plugs of Achlya bisexualis, and also showed inhibition (FIG. 15B).

### Example 17

### Identification and Bioactivity Assays of the Compound 4-Cumylphenol

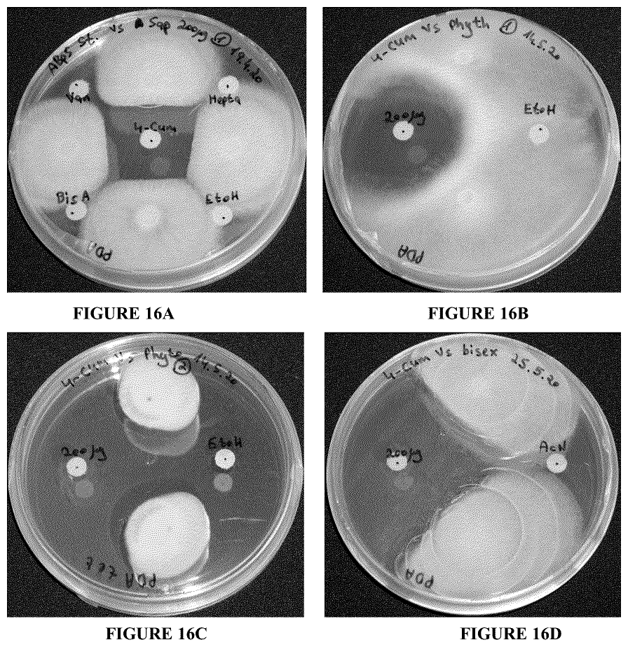

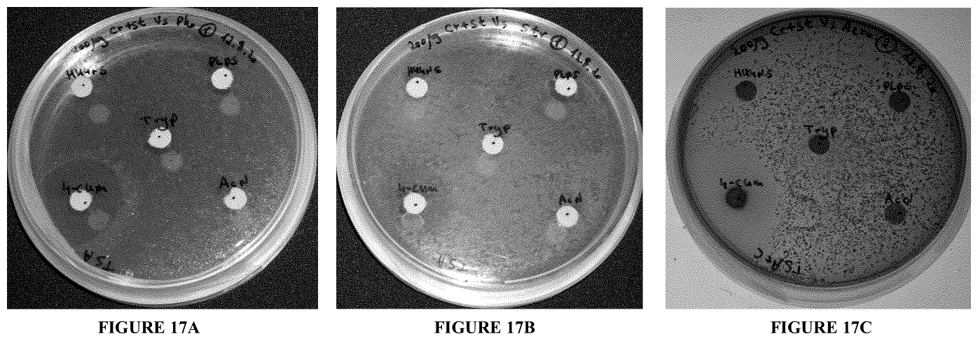

The compound 4-Cumylphenol was identified in a liquid crude extract of ABp5 by using HP-LC analysis for active fraction separation and GC-MS analysis for metabolite identifications. An available authentic standard was purchased (Alfa Aesar, United states) and tested in-vitro against S. parasitica (FIG. 16A). 4-Cumylphenol was tested against other pathogenic bacteria and oomycete. Two hundred (200) μg of 4-Cumylphenol were added on a paper disk and introduced to PDA petri plates together with Pythium aphanidermatum (FIG. 16B), Phytophthora infestans (FIG. 16C), Achlya bisexualis (FIG. 16D) plugs, and showed inhibition of the oomycete mycelium. Two hundred (200) μg of 4-Cumylphenol were tested against the aquatic pathogenic bacteria Photobacterium damselae (FIG. 17A), Streptococcus iniae (FIG. 17B) and Aeromonas salmonicida (FIG. 17C) as well and showed inhibitory activity in in-vitro assays.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An artificial composition comprising cultured microorganisms comprising at least one isolate listed in Table 2, and an agriculturally acceptable carrier.

2. The artificial composition of claim 1, further comprising one or more metabolites produced by said at least one isolate, secreted therefrom, or both, optionally wherein said at least one isolate is selected from the group consisting of: AJr9, ABp5, PU9, PU10, HU4N5, AU4, AJr10, PSH6, PSH7, ABp4, PLp5, PP2, PP6, KM3, KM4, PAn4, PAc1, HGGCy9, HGM1, HG3N2, TAPaN3, TAEnN2, and TAAnN2, and optionally wherein said at least one isolate is AJr9.

3-4. (canceled)

5. The artificial composition of claim 1, further comprising one or more bacteria belonging to a genus selected from the group consisting of: Bacillus, Lysinibacillus, Pseudomonas, Staphylococcus, and any combination thereof.

6. The artificial composition of claim 1, further comprising at least one fungus belonging to the genus Beauveria.

7. The artificial composition of claim 1, further comprising an alga.

8. The artificial composition of claim 1, being a dried or a lyophilized composition.

9. The artificial composition of claim 1, characterized by being capable of inhibiting a pathogen's growth, killing a pathogen, inhibiting the secretion, activity, or both, of metabolites derived from a pathogen, or any combination thereof, and optionally wherein said pathogen is a microorganism.

10. (canceled)

11. The artificial composition of claim 9, wherein said pathogen is selected from the group consisting of: a bacterium, a fungus, and an oomycete.

12. The artificial composition of claim 9, wherein said pathogen is selected from the group consisting of: Photobacterium damselae subsp. Damselae, Streptococcus iniae, Aeromonas salmonicida, Saprolegnia parasitica, and any combination thereof.

13. The artificial composition of claim 12, wherein said pathogen is Photobacterium damselae subsp. Damselae and said at least one isolate is selected from the group consisting of: AJr3, AJr7, AJr9, AJr10, AJr15, PSH1, PSH3, PSH5, PSH6, ABp1, ABp2, ABp3, ABp4, PAc1, PAc2, TAAcN4, PLp2, PLp5, PLp6, PAn4, TAAnN1, TAAnN2, TAAnN3, PP1, PP2, PP3, PP6, PP7, PP8, TAPaN1, TAPaN2, TAPaN3, KM FWTA1, KM FWTA3, HG9, PU9, PU10, AU1, HU4N5, HG2N7, HG3N2, HG3M1, TAEnN2, TAEnN4, and any combination thereof.

14. The artificial composition of claim 12, wherein said pathogen is Streptococcus iniae and said at least one isolate is selected from the group consisting of: AJr3, AJr9, AJr10, AJr15, PSH1, PSH3, PSH5, PSH6, ABp1, ABp2, PAc1, PAc2, TAAcN4, PLp2, PLp5, PLp6, PAn4, TAAnN1, TAAnN2, TAAnN3, PP1, PP2, PP3, PP6, PP7, PP8, TAPaN1, TAPaN2, TAPaN3, KM FWTA1, KM FWTA3, FWTa5, PU9, PU10, AU1, AU4, HU4N5, HG2N7, HG3N2, HG3M1, and any combination thereof.

15. The artificial composition of claim 12, wherein said pathogen is Aeromonas salmonicida and said at least one isolate is selected from the group consisting of: AJr3, AJr7, AJr9, AJr10, PSH1, PSH3, PSH5, PSH6, PSH7, ABp1, ABp2, ABp3, ABp4, PAc1, PAc2, TAAcN4, PLp2, PLp5, PLp6, PAn4, TAAnN1, TAAnN2, TAAnN3, PP2, PP3, PP6, PP7, TAPaN1, TAPaN2, TAPaN3, KM FWTA1, KM FWTA3, FWTa1, FWTa5, HG9, PU10, AU1, AU4, HU4N5, HG2N7, HG3N2, HG3M1, TAEnN2, TAEnN4, and any combination thereof.

16. The artificial composition of claim 12, wherein said pathogen is Saprolegnia parasitica and said at least one isolate is selected from the group consisting of: AJr3, AJr7, AJr9, AJr10, AJr15, PSH1, PSH3, PSH5, PSH6, PSH7, ABp1, ABp2, ABp3, ABp4, ABp5, PAc1, PAc2, TAAcN4, PLp2, PLp5, PLp6, PP2, PP6, PP7, TAPaN3, TAPaN3, KM FWTA1, KM FWTA3, FWTA4, PU9, PU10, AU1, AU4, HG3M1, TAEnN2, and any combination thereof.

17. The artificial composition of claim 1, being a feed composition, optionally wherein said feed is suitable for an aquatic organism, and optionally wherein said aquatic organism is an aquacultured organism.

18.-19. (canceled)

20. The artificial composition of claim 2, wherein said one or more metabolites is 8-Nonenoic acid, 4-cumylphenol, or a combination thereof.

21. A method for preventing or treating a pathogenic infection in an aquatic organism, the method comprising contacting said aquatic organism with an effective amount of an artificial composition comprising cultured microorganisms comprising at least one isolate listed in Table 2, and an agriculturally acceptable carrier, optionally wherein said aquatic organism is cultured in freshwater, brackish water, or saline water.

22. (canceled)

23. The method of claim 21, wherein said organism is selected from the group consisting of: a fish, a crustacean, a mollusk, a macro-alga, a phytoplankton, phytoplankton, and zooplankton.

24. The method of claim 21, wherein said contacting comprises contacting a body of water comprising said aquatic organism.

25. The method of claim 21, wherein said contacting comprises providing said artificial composition as feed to said aquatic organism.

26. The method of claim 25, wherein said feed comprises said artificial composition, and optionally wherein any one of: (i) said feed is suitable for an aquatic organism: (ii) said aquatic organism is an aquacultured organism: (iii) said artificial composition further comprises one or more metabolites produced by said at least one isolate, secreted therefrom, or both: (iv) said one or more metabolites is 8-Nonenoic acid, 4-cumylphenol, or a combination thereof; and (v) any combination of (i) to (iv).
